(19) United States
(12) Patent Application Publication
(10) Pub. No.: US 2022/0242944 A1
(43) Pub. Date: Aug. 4, 2022

(54) ANTIGEN BINDING MOLECULES THAT BIND PDGF-B AND PDGF-D AND USES THEREOF

(71) Applicant: Chugai Seiyaku Kabushiki Kaisha, Tokyo (JP)

(72) Inventors: Masakazu KANAMORI, Singapore (SG); Hideaki SHIMADA, Singapore (SG)

(21) Appl. No.: 17/621,746

(22) PCT Filed: Jun. 26, 2020
(86) PCT No.: PCT/JP2020/025135

§ 371 Date: Dec. 22, 2021

(30) Foreign Application Priority Data
Jun. 28, 2019 (JP) 2019-120991

(51) Int. Cl. C07K 16/22

(52) U.S. Cl. CPC C07K 16/22; C07K 2317/31; C07K 2317/52; C07K 2317/565; C07K 2317/56; C07K 2317/24

(57) ABSTRACT

The present invention provides antigen-binding molecules or antibodies which specifically bind to PDGF-B and/or PDGF-D. The invention further relates to compositions and therapeutic methods for using these antigen-binding molecules or antibodies for the treatment and/or prevention of PDGF-mediated diseases, disorders, or conditions.

### TECHNICAL FIELD

The present invention relates to antigen-binding molecules that bind to PDGF-B and/or PDGF-D, pharmaceutical compositions comprising the same, and methods of using the same.

### BACKGROUND ART

Many chronic diseases are characterized by persistent and sustained inflammation, injury, tissue remodeling, and fibrosis. For example, progressive renal diseases, which include diabetic nephropathy, IgA nephropathy, and proliferative lupus nephritis, are histologically characterized by mesangial cell expansion and glomerular and tubulointerstitial fibrosis.

Platelet-derived growth factor (PDGF)-signaling is one of the central mediators involved in fibrosis. Stromal mesenchymal cells express both PDGF receptor (PDGFR) alpha and beta, activation of which drives proliferation and migration of cells, and production of extracellular matrix, i.e. the principal processes of fibrosis (NPL1). PDGFs have also been implicated in a wide variety of human diseases, including, but not limited to, atherosclerosis, restenosis, pulmonary hypertension, retinal vascular disease, organ fibrosis (e.g., cardiac, lung, liver and kidney), rheumatoid arthritis, osteoarthritis, tumorigenesis, and systemic sclerosis (SSc; scleroderma) (NPL2-5).

PDGF family includes five isoforms: PDGF-AA, -AB, -BB, -CC, and -DD, binding to the tyrosine kinase PDGF receptor (PDGFR) dimers alpha-alpha, alpha-beta, or beta-beta. PDGF-A and -C predominantly bind to the PDGFR-alpha chain, PDGF-B binds to both -alpha and -beta chains, whereas PDGF-D binds to the -beta chain only (NPL6). Upon ligand binding, PDGFRs become phosphorylated and interact and activate various cytoplasmic downstream signaling pathways and transcription factors, e.g. phospholipase C, ras GTPase activating protein, phosphatidyl inositol 3-kinase (PI3K), Janus kinase, mitogen activated protein kinase, p38, or calcium release, which drive gene expression and cellular effects of PDGF (NPL7).

Fibrosis is a hallmark of pathologic remodeling in numerous tissues and a contributor to clinical diseases. Virtually all chronic progressive diseases are associated with fibrosis, representing a huge number of patients worldwide. Although we understand many of the cellular and molecular processes underlying fibrosis, there are few effective treatment options (NPL8). Therefore, there is a long-felt need for novel potential therapeutics that can effectively treat or ameliorate these diseases/disorders, and the present invention meets this need.

### CITATION LIST

### Non Patent Literature

[NPL1] B. M. Klinkhammer et al. Molecular Aspects of Medicine 62 (2018) 44-62
[NPL2] Trojanowska, 2008, Rheumatology 47:v2-v4
[NPL3] Andrae et al. 2008 Genes Dev. 22: 1276-1312.
[NPL4] Ying et al. Mol Med Rep. 2017 December; 16(6): 7879-7889
[NPL5] P. Boor et al. Nephrology Dialysis Transplantation, Volume 29, February 2014 45-54
[NPL6] Chen et al. Biochim Biophys Acta. 2013 October; 1834(10): 2176-2186.
[NPL7] Heldin Cell Communication and Signaling 2013 11:97
[NPL8] Don C. Rockey et al. N Engl J Med 2015; 372:1138-1149

### SUMMARY OF INVENTION

### Technical Problem

An objective of the present invention is to provide antigen-binding molecules which effectively inhibit PDGF-mediated diseases or disorders such as fibrotic diseases or fibrosis.

### Solution to Problem

The present inventors have conducted diligent studies and have successfully prepared antigen-binding molecules, e.g., antibodies and antigen binding fragments thereof, that specifically bind to platelet-derived growth factor B (PDGF-B) and/or platelet-derived growth factor D (PDGF-D). The invention further relates to compositions comprising a multispecific antibody binding to PDGF-B and PDGF-D, and methods of using the antibodies as a medicament. It is known that the PDGF family is composed of four different polypeptide chains PDGF-A, B, C, and D (forming PDGF-AA, CC, AB, BB, and DD dimers), each of which is capable of mediating PDGF signaling by activating tyrosine kinase receptors PDGF-R alpha and beta. The present inventors have found that the dual blockade of PDGF-B and PDGF-D out of the five isoforms of PDGF (AA, CC, AB, BB, and DD) is sufficient to effectively inhibit or treat PDGF-mediated diseases or disorders such as fibrosis. Significantly, the present inventors have prepared antibodies binding and inhibiting both PDGF-B and PDGF-D, and have demonstrated that the dual PDGF-B and PDGF-D binding antibodies have combined inhibitory effect and are effective in inhibiting, preventing, or treating PDGF-mediated diseases or disorders.

More specifically, the present invention relates to:
[1] A pharmaceutical composition comprising an antigen-binding molecule which binds to PDGF-B and an antigen-binding molecule which binds to PDGF-D.
[1A] A pharmaceutical composition comprising an antigen-binding molecule which binds to PDGF-B in combination with a pharmaceutical composition comprising an antigen-binding molecule which binds to PDGF-D.
[1B] A pharmaceutical composition comprising an antigen-binding molecule which binds to PDGF-D in combination with a pharmaceutical composition comprising an antigen-binding molecule which binds to PDGF-B.
[1C] A combination medicine comprising a pharmaceutical composition comprising an antigen-binding molecule which binds to PDGF-B and an antigen-binding molecule which binds to PDGF-D.
[1D] The pharmaceutical composition of any one of [1] to [1C], wherein the antigen-binding molecule which binds to PDGF-B is administered to a subject simultaneously, separately, or sequentially with the antigen-binding molecule which binds to PDGF-D.
[2] A multispecific antigen-binding molecule comprising a first antigen-binding domain that binds to PDGF-B, and a second antigen-binding domain that binds to PDGF-D.
[3] The multispecific antigen-binding molecule of [2], wherein the antigen-binding molecule has one or more of the following properties:
   i) inhibiting PDGF-B and PDGF-D binding to PDGFR alpha and/or PDGFR beta;
   ii) inhibiting PDGF-B and PDGF-D mediated phosphorylation of PDGFR alpha and/or PDGFR beta;
   iii) inhibiting PDGF-B and PDGF-D induced dimerization of PDGFR alpha and/or PDGFR beta;
   iv) inhibiting PDGF-B and PDGF-D induced mitogenesis of cells displaying PDGFR alpha and/or PDGFR beta; and
   v) not binding to PDGF-A and/or PDGF-C.
[4] The antigen-binding molecule of any one of [2] to [3], wherein the antigen-binding molecule is an antibody, preferably a monoclonal antibody, a chimeric antibody, a humanized antibody, a human antibody, or a fragment thereof.
[5] The antigen-binding molecule of any one of [2] to [4], wherein the first antigen-binding domain that binds to PDGF-B is:
   (a) an antigen-binding domain that comprises a VH comprising the amino acid sequence of SEQ ID NO: 1; and a VL comprising the amino acid sequence of SEQ ID NO: 2;
   (b) an antigen-binding domain that comprises a VH comprising the CDR-H1 amino acid sequence of SEQ ID NO: 5, the CDR-H2 amino acid sequence of SEQ ID NO: 6, and the CDR-H3 amino acid sequence of SEQ ID NO: 7; and a VL comprising the CDR-L1 amino acid sequence of SEQ ID NO: 8, the CDR-L2 amino acid sequence of SEQ ID NO: 9, and the CDR-L3 amino acid sequence of SEQ ID NO: 10;
   (c) an antigen-binding domain that binds to the same epitope on PDGF-B with any one of the antigen-binding domains of (a) to (b); or
   (d) an antigen-binding domain that competes for binding to PDGF-B with any one of the antigen-binding domains of (a) to (b);
      and/or
      the second antigen-binding domain that binds to PDGF-D is:
   (e) an antigen-binding domain that comprises a VH comprising the amino acid sequence of SEQ ID NO: 3; and a VL comprising the amino acid sequence of SEQ ID NO: 4;
   (f) an antigen-binding domain that comprises a VH comprising the CDR-H1 amino acid sequence of SEQ ID NO: 11, the CDR-H2 amino acid sequence of SEQ ID NO: 12, and the CDR-H3 amino acid sequence of SEQ ID NO: 13; and a VL comprising the CDR-L1 amino acid sequence of SEQ ID NO: 14, the CDR-L2 amino acid sequence of SEQ ID NO: 15, and the CDR-L3 amino acid sequence of SEQ ID NO: 16;
   (g) an antigen-binding domain that binds to the same epitope on PDGF-D with any one of the antigen-binding domains of (e) to (f); or
   (h) an antigen-binding domain that competes for binding to PDGF-D with any one of the antigen-binding domains of (e) to (1).
[6] The antigen-binding molecule of any one of [2] to [5], further comprises an antibody Fc region with reduced binding activity towards an Fc gamma receptor.
[7] The pharmaceutical composition of any one of [1] to [1D], or the antigen-binding molecule of any one of [2] to [6], for use in the treatment of a fibrotic disease or fibrosis.
[7A] Use of the pharmaceutical composition of any one of [1] to [1D], or the antigen-binding molecule of any one of [2] to [6], for the manufacture of a medicament for the treatment of a fibrotic disease or fibrosis.
[8] A method for preventing, treating, or inhibiting a fibrotic disease or fibrosis comprising: administering to a mammalian subject suffering from the fibrotic disease or fibrosis the pharmaceutical composition of any one of [1] to [1D], or the antigen-binding molecule of any one of [2] to [6].
[8A] A method for preventing or treating a disease, disorder, or condition mediated by the PDGF-B binding to PDGFR, said method comprising administering to a subject in need thereof an effective amount of the pharmaceutical composition of any one of [1] to [1D], or the antigen-binding molecule of any one of [2] to [6].
[8B] The method of [8A], wherein said disease, disorder, or condition is at least one selected from the group consisting of: fibrosis (such as myocardial fibrosis, pulmonary fibrosis, liver fibrosis, renal fibrosis, skin fibrosis, ocular fibrosis and myelofibrosis), nephritis and related diseases in humans including but not limited to, nephritis, progressive renal diseases, and related diseases, such as IgA nephropathy, mesangial proliferative nephritis, mesangial proliferative glomerulonephritis, mesangiocapillary glomerulonephritis, systemic lupus erythematosus, glomerular nephritis, renal interstitial fibrosis, renal failure, diabetic nephropathy, polycystic kidney disease, alport syndrome, focal segmental glomerular sclerosis, and membranous nephropathy.
[9] The pharmaceutical composition or antigen-binding molecule for use, the use, or the method according to any one of [5]-[8], wherein the fibrotic disease or fibrosis is characterized by upregulated PDGF signaling activation.
[10] The pharmaceutical composition or antigen-binding molecule for use, the use, or the method according to any one of [5]-[8] and [9], wherein the fibrotic disease or fibrosis is myocardial fibrosis, pulmonary fibrosis, liver fibrosis, renal fibrosis, skin fibrosis, ocular fibrosis and myelofibrosis, nephritis, progressive renal diseases, IgA nephropathy, mesangial proliferative nephritis, mesangial proliferative glomerulonephritis, mesangiocapillary glomerulonephritis, systemic lupus erythematosus, glomerular nephritis, renal interstitial fibrosis, renal failure, diabetic nephropathy, polycystic kidney disease, alport syndrome, focal segmental glomerular sclerosis, or membranous nephropathy.
[11] The pharmaceutical composition or antigen-binding molecule for use, the use, or the method according to any one of [5]-[8] and [9], wherein the fibrotic disease or fibrosis is kidney fibrosis, preferably characterized by having interstitial fibrosis or glomerulosclerosis.
[11A] The pharmaceutical composition or antigen-binding molecule for use, the use, or the method according to any one of [5]-[8] and [9], wherein the fibrotic disease or fibrosis is liver fibrosis or nonalcoholic steatohepatitis (NASH).
[11B] The pharmaceutical composition or antigen-binding molecule for use, the use, or the method according to any one of [5] to [11A], wherein the subject is human.
[12] An isolated polynucleotide comprising a nucleotide sequence that encodes the antigen-binding molecule of any one of [1] to [6].
[13] An expression vector comprising the polynucleotide according to [12].
[14] A host cell transformed or transfected with the polynucleotide according to [12] or the expression vector according to [13].
[15] A method of producing an antigen-binding molecule comprising:
   (a) identifying one or more antigen-binding domain that binds to PDGF-B;
   (b) identifying one or more antigen-binding domain that binds to PDGF-D; and
   (c) preparing an antigen-binding molecule comprising the antigen-binding domain identified in (a) and (b).
[16] The method of [15], further comprising one or more of the following steps:
   (a) identifying one or more antigen-binding domain that has one or more of the following properties
      i) inhibiting PDGF-B binding to PDGFR alpha and/or PDGFR beta;
      ii) inhibiting PDGF-B mediated phosphorylation of PDGFR alpha and/or PDGFR beta;
      iii) inhibiting PDGF-B induced dimerization of PDGFR alpha and/or PDGFR beta;
      iv) inhibiting PDGF-B induced mitogenesis of cells displaying PDGFR alpha and/or PDGFR beta;
      v) not binding to PDGF-A and/or PDGF-C; and
   (b) identifying one or more antigen-binding domain that has one or more of the following properties
      i) inhibiting PDGF-D binding to PDGFR alpha and/or PDGFR beta;
      ii) inhibiting PDGF-D mediated phosphorylation of PDGFR alpha and/or PDGFR beta;
      iii) inhibiting PDGF-D induced dimerization of PDGFR alpha and/or PDGFR beta;
      iv) inhibiting PDGF-D induced mitogenesis of cells displaying PDGFR alpha and/or PDGFR beta;
      v) not binding to PDGF-A and/or PDGF-C.
[17] The antigen-binding molecule of any one of [15] to [16], wherein the antigen-binding molecule is an antibody, preferably a monoclonal antibody, a chimeric antibody, a humanized antibody, a human antibody, or a fragment thereof.

In another aspect, the present invention further relates to an antigen-binding molecule that specifically binds to PDGF-D and blocks its interaction with Neuropilin 1 (NRP1). NRP1 binds to PDGF-D and is a co-receptor in PDGF-D-PDGFR-beta signalling (Muhl, Lars, et al., J Cell Sci 130.8 (2017): 1365-1378). In one embodiment, the antigen-binding molecule is an antibody that specifically binds to PDGF-D and blocks/inhibits its interaction with Neuropilin 1 (NRP1) and also blocks/inhibits PDGF-D binding to PDGFR, thereby inhibits the PDGF-D-induced signalling. Such antibody is expected to show enhanced inhibition of PDGF-D-mediated signalling compared to anti-PDGF-D antibody that is not capable of blocking NRP1-PDGF-D interaction, for use as a more effective anti-PDGF-D antibody for treating/preventing a PDGF-D-mediated disease/condition. Method of obtaining an antibody that specifically binds to PDGF-D and blocks/inhibits its interaction with NRP1 and also blocks/inhibits PDGF-D binding to PDGFR include known antigen immunization followed by evaluation and screening for inhibition of NRP1-PDGF-D interaction using well-known method such as ELISA, Octet, Biacore and/or ECL and so on.

### BRIEF DESCRIPTION OF DRAWINGS

FIG. 1 shows the results of evaluating phosphorylation of PDGFR beta in mouse fibroblast cell line NIH3T3. CPR is an anti-PDGF-B antibody, CR is an anti-PDGF-D antibody, and CPR//CR is an anti-PDGF-B and -D bispecific antibody.

FIG. 2 shows the results of evaluating phosphorylation of PDGFR beta in human fibroblast cell line IMR90. CPR is an anti-PDGF-B antibody, CR is an anti-PDGF-D antibody, and CPR//CR is an anti-PDGF-B and -D bispecific antibody.

FIG. 3 shows the results of evaluating phosphorylation of PDGFR alpha in human fibroblast cell line IMR90. CPR is an anti-PDGF-B antibody, CR is an anti-PDGF-D antibody, and CPR//CR is an anti-PDGF-B and -D bispecific antibody.

FIG. 4 shows the results of BrdU cell proliferation assay in mouse fibroblast cell line NIH3T3. CPR is an anti-PDGF-B antibody, CR is an anti-PDGF-D antibody, and CPR//CR is an anti-PDGF-B and -D bispecific antibody.

FIG. 5 shows the results of evaluating phosphorylation of PDGFR beta in human fibroblast cell line IMR90. IC17 is an anti-KLH antibody used as a negative control. CR is an anti-PDGF-D antibody. NRP1-Fc is a recombinant protein in which human neuropilin-1 ECD and Fc region of human IgG1 are fused.

FIG. 6 shows the results of BrdU cell proliferation assay in human fibroblast. CR is an anti-PDGF-D antibody. NRP1-Fc is a recombinant protein in which human neuropilin-1 ECD and Fc region of human IgG1 are fused.

FIG. 7A shows the results of evaluating collagen type 1 alpha 1 (Collal) mRNA levels in kidney. Efficacy of monoclonal antibodies was evaluated in Unilateral Ureteral Obstruction (UUO) induced mouse renal fibrosis model. Sham operated group represents as non-disease-induced control. Collal mRNA was suppressed by treatment with anti-PDGF antibodies. IC17 is an anti-KLH antibody used as a negative control. CPR001 is an anti-PDGF-B antibody. CR002 is an anti-PDGF-D antibody.

FIG. 7B shows the results of evaluating hydroxyproline content in kidney. Efficacy of monoclonal antibodies was evaluated in Unilateral Ureteral Obstruction (UUO) induced mouse renal fibrosis model. Sham operated group represents as non-disease-induced control. Kidney fibrosis was reduced by treatment with anti-PDGF antibodies. IC17 is an anti-KLH antibody used as a negative control. CPR001 is an anti-PDGF-B antibody. CR002 is an anti-PDGF-D antibody.

FIG. 8A shows the results from plasma creatinine concentration measurements. The top panel shows the time-course changes in plasma creatinine concentration. The bottom panel shows the results of plasma creatinine at week 20. Efficacy of monoclonal antibodies was evaluated in Alport mouse chronic kidney disease (CKD) model (Col4a3 KO mouse). Wild type represents as non-disease-induced control (C57BL/6J). Plasma creatinine was suppressed by treatment with anti-PDGF antibodies. IC17 is an anti-KLH antibody used as a negative control. CPR001 is an anti-PDGF-B antibody. CR002 is an anti-PDGF-D antibody.

FIG. 8B shows the results from plasma cystatin C concentration measurements. The top panel shows the time-course changes in plasma cystatin C concentration. The bottom panel shows the results of plasma cystatin C measurement at week 20. Efficacy of monoclonal antibodies was evaluated in Alport mouse chronic kidney disease (CKD) model (Col4a3 KO mouse). Wild type represents as non-disease-induced control (C57BL/6J). Plasma cystatin C was suppressed by treatment with anti-PDGF antibodies. IC17 is an anti-KLH antibody used as a negative control. CPR001 is an anti-PDGF-B antibody. CR002 is an anti-PDGF-D antibody.

FIG. 8C shows the results of evaluating collagen type 1 alpha 1 (Collal) mRNA levels in kidney. Efficacy of monoclonal antibodies was evaluated in Alport mouse chronic kidney disease (CKD) model (Col4a3 KO mouse). Wild type group represents as non-disease-induced control. Collal mRNA was suppressed by treatment with anti-PDGF antibodies. IC17 is an anti-KLH antibody used as a negative control. CPR001 is an anti-PDGF-B antibody. CR002 is an anti-PDGF-D antibody.

FIG. 8D shows the results of evaluating hydroxyproline content in kidney. Efficacy of monoclonal antibodies was evaluated in Alport mouse chronic kidney disease (CKD) model (Col4a3 KO mouse). Wild type group represents as non-disease-induced control. Kidney fibrosis was reduced by treatment with anti-PDGF antibodies. IC17 is an anti-KLH antibody used as a negative control. CPR001 is an anti-PDGF-B antibody. CR002 is an anti-PDGF-D antibody.

FIG. 9A shows the results of evaluating collagen type 1 alpha 1 mRNA levels in liver. Efficacy of monoclonal antibodies was evaluated in choline-deficient, L-amino acid-defined, high-fat diet (CDAHFD) induced mouse NASH/liver fibrosis model. Normal diet group represents as non-disease-induced control. IC17 is an anti-KLH antibody used as a negative control. CPR is an anti-PDGF-B Ab, CR is an anti-PDGF-D antibody (CR002 as described in WO2007059234). Combi group is treated with CPR and CR.

FIG. 9B shows the results of evaluating hydroxyproline content in liver. Efficacy of monoclonal antibodies was evaluated in choline-deficient, L-amino acid-defined, high-fat diet (CDAHFD) induced mouse NASH/liver fibrosis model. Normal diet group represents as non-disease-induced control. IC17 is an anti-KLH antibody used as a negative control. CPR is an anti-PDGF-B antibody. CR is an anti-PDGF-D antibody (CR002 as described in WO2007059234). Combi group is treated with CPR and CR. Liver fibrosis was reduced by treatment with anti-PDGF antibodies.

FIG. 10 shows the results of evaluating collagen type 1 alpha 1 (Collal) mRNA levels in kidney. Efficacy of monoclonal antibodies were evaluated in Unilateral Ureteral Obstruction (UUO) induced mouse renal fibrosis model. Sham operated group represents as non-disease-induced control. Collal mRNA was suppressed by treatment with CPR//CR. IC17 is an anti-KLH antibody used as a negative control. CPR//CR is a bi-specific antibody against PDGF-B and PDGF-D.

FIG. 11 shows the results of evaluating hydroxyproline content in kidney. Efficacy of monoclonal antibodies were evaluated in Unilateral Ureteral Obstruction (UUO) induced mouse renal fibrosis model. Sham operated group represents as non-disease-induced control. Kidney fibrosis was reduced by treatment with CPR//CR. IC17 is an anti-KLH antibody used as a negative control. CPR//CR is a bi-specific antibody against PDGF-B and PDGF-D.

FIG. 12 shows the results from plasma creatinine concentration measurements. The time-course changes in plasma creatinine concentration is shown in A and the results of plasma creatinine at week 20 is shown in B. Monoclonal antibodies were evaluated in Alport mouse chronic kidney disease (CKD) model (Col4a3 KO mouse). Wild type represents as non-disease-induced control (C57BL/6J). Plasma creatinine was suppressed by treatment with CPR//CR. IC17 is an anti-KLH antibody used as a negative control. CPR//CR is a bi-specific antibody against PDGF-B and PDGF-D.

FIG. 13 shows the result of evaluating collagen type 1 alpha 1 (Collal) mRNA levels in kidney. Efficacy of monoclonal antibodies were evaluated in Alport mouse chronic kidney disease (CKD) model (Col4a3 KO mouse). Wild type group represents as non-disease-induced control. Collal mRNA was suppressed by treatment with CPR//CR. IC17 is an anti-KLH antibody used as a negative control. CPR//CR is a bi-specific antibody against PDGF-B and PDGF-D.

FIG. 14 shows the result of evaluating hydroxyproline content in kidney. Efficacy of monoclonal antibodies was evaluated in Alport mouse chronic kidney disease (CKD) model (Col4a3 KO mouse). Wild type group represents as non-disease-induced control. Kidney fibrosis was reduced by treatment with CPR//CR. IC17 is an anti-KLH antibody used as a negative control. CPR//CR is a bi-specific antibody against PDGF-B and PDGF-D.

FIG. 15 shows the results of evaluating collagen type 1 alpha 1 mRNA in liver. Efficacy of monoclonal antibody were evaluated in choline-deficient, L-amino acid-defined, high-fat (CDAHFD) induced mouse NASH/liver fibrosis model. Normal diet group represents as non-disease control. Collal mRNA was suppressed by treatment with CPR//CR. IC17 is an anti-KLH antibody used as a negative control. CPR//CR is a bi-specific antibody against PDGF-B and PDGF-D.

FIG. 16 shows the results of evaluating plasma aspartate aminotransferase (AST) and alanine aminotransferase (ALT). Efficacy of monoclonal antibodies were evaluated in choline-deficient, L-amino acid-defined, high-fat (CDAHFD) induced mouse NASH/liver fibrosis model. Plasma AST (upper panel) and ALT (lower panel) were suppressed by treatment with CPR//CR. Normal diet group represents as non-disease control. IC17 is an anti-KLH antibody used as a negative control. CPR//CR is a bi-specific antibody against PDGF-B and PDGF-D.

FIG. 17 is a concentration response curve showing competition binding of anti-PDGF-D antibody (CR002) and hPDGFR beta against hPDGF-D as evaluated with premix competition assay. Binding of PDGF-D to PDGFR beta was blocked with increased concentration of anti-PDGF-D antibody (CR002).

FIG. 18 shows the results from Biacore in-tandem blocking assay to characterize binding epitope of anti-PDGF-D antibody (CR002) and hNRP1-Fc on hPDGF-D. A binding response for CR002 injection which was greater than that observed for buffer injection indicates that CR002 and hNRP-1 bind to different epitopes on hPDGF-D.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

The definitions and detailed description below are provided to facilitate understanding of the present invention illustrated herein.

### Amino Acids

Herein, amino acids are described by three- or one-letter codes or both, for example, Ala/A for alanine, Leu/L for leucine, Arg/R for arginine, Lys/K for lysine, Asn/N for asparagine, Met/M for methionine, Asp/D for aspartic acid, Phe/F for phenylalanine, Cys/C for cysteine, Pro/P for proline, Gln/Q for glutamine, Ser/S for serine, Glu/E for glutamic acid, Thr/T for threonine, Gly/G for glycine, Trp/W for tryptophan, His/H for histidine, Tyr/Y for tyrosine, Ile/I for isoleucine, or Val/V for valine.

### Alteration of Amino Acids

For amino acid alteration in the amino acid sequence of an antigen-binding molecule, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is suitable to use a cell-free translation system (Clover Direct (Protein Express)) containing a tRNA which has a non-natural amino acid bound to a complementary amber suppressor tRNA of one of the stop codons, the UAG codon (amber codon).

In the present specification, the meaning of the term “and/or” when describing the site of amino acid alteration includes every combination where “and” and “or” are suitably combined. Specifically, for example, “the amino acids at positions 33, 55, and/or 96 are substituted” includes the following variation of amino acid alterations: amino acid alteration(s) at (a) position 33, (b) position 55, (c) position 96, (d) positions 33 and 55, (e) positions 33 and 96, (f) positions 55 and 96, and (g) positions 33, 55, and 96.

Furthermore, herein, as an abbreviated expression showing a specific alteration of an amino acid, an expression that combines a number (indicating the position of the altered amino acid) and one-letter or three-letter amino acid codes (indicating the amino acids before and after the alteration) may be used appropriately, which expression may be organized in the following order: code for the amino acid before the alteration—number indicating the position—code for the amino acid after the alteration. For example, the expression N100bL or Asn100bLeu used to refer to a substitution of an amino acid contained in an antibody variable region indicates substitution of Asn at position 100b (according to Kabat numbering) with Leu. That is, the number shows the amino acid position according to Kabat numbering, the one-letter or three-letter amino-acid code written before the number shows the amino acid before substitution, and the one-letter or three-letter amino-acid code written after the number shows the amino acid after substitution. Similarly, the alteration P238D or Pro238Asp used to refer to a substitution of an amino acid of the Fc region contained in an antibody constant region indicates substitution of Pro at position 238 (according to EU numbering) with Asp. That is, the number shows the amino acid position according to EU numbering, the one-letter or three-letter amino-acid code written before the number shows the amino acid before substitution, and the one-letter or three-letter amino-acid code written after the number shows the amino acid after substitution.

### Antigen-Binding Molecule

The term “antigen-binding molecule”, as used herein, refers to any molecule that comprises an antigen-binding domain or any molecule that has binding activity to an antigen, and may further refer to molecules such as a peptide or protein having a length of about five amino acids or more. The peptide and protein are not limited to those derived from an organism, and for example, may be a polypeptide produced from an artificially designed sequence. They may also be any of a naturally-occurring polypeptide, synthetic polypeptide, recombinant polypeptide, and such.

A favorable example of an antigen-binding molecule of the present invention is an antigen-binding molecule that comprises a plurality of antigen-binding domains. In certain embodiments, the antigen-binding molecule of the present invention comprises two antigen-binding domains with different antigen-binding specificities. In certain embodiments, the antigen-binding molecule of the present invention comprises two antigen-binding domains with different antigen-binding specificities, and an FcRn-binding domain contained in an antibody Fc region. As a method for extending the blood half-life of a protein administered to a living body, the method of adding an FcRn-binding domain of an antibody to the protein of interest and utilizing the function of FcRn-mediated recycling is well known.

### Antigen-Binding Domain

The term “antigen-binding domain”, as used herein, refers to a portion of an antibody which portion comprises a region that specifically binds and is complementary to the whole or a portion of an antigen. When the molecular weight of an antigen is large, an antibody can only bind to a particular portion of the antigen. The particular portion is called “epitope”. An antigen-binding domain can be provided from one or more antibody variable domains. Preferably, the antigen-binding domains contain an antibody variable region that comprises both an antibody light chain variable region (VL) and an antibody heavy chain variable region (VH). Such preferable antigen-binding domains include, for example, “single-chain Fv (scFv)”, “single-chain antibody”, “Fv”, “single-chain Fv₂ (scFv₂)”, “Fab”, and “F (ab′)₂”.

The antigen-binding domains of an antigen-binding molecule of the present invention specifically bind to PDGF-B and/or PDGF-D. That is, PDGF-B and PDGF-D are respectively preferable antigens of interest—the antigen-binding domain has binding activity to its antigen of interest but does not substantially recognize and bind to other molecules in a sample containing a mixed population of antigenic molecules. As used herein, the phrase “has binding activity” refers to the activity of an antigen-binding domain, antibody, antigen-binding molecule, antibody variable fragment, or such (hereinafter, “antigen-binding domain or such”) to bind to an antigen of interest at a level of specific binding, higher than the level of non-specific or background binding. In other words, such an antigen-binding domain or such “has a specific/significant binding activity” towards the antigen of interest. The specificity can be measured by any methods for detecting affinity or binding activity as described herein or known in the art. The above-mentioned level of specific binding may be high enough to be recognized by a skilled person as being significant. For example, when a skilled person can detect or observe any significant or relatively strong signals or values of binding between the antigen-binding domain or such and the antigen of interest in a suitable binding assay, it can be said that the antigen-binding domain or such has a “specific/significant binding activity” towards the antigen of interest. Alternatively, “have a specific/significant binding activity” can be rephrased as “specifically/significantly bind” (to the antigen of interest). Sometimes, the phrase “having binding activity” has substantially the same meaning as the phrase “having a specific/significant binding activity” in the art. As used herein, an antigen-binding molecule or an antibody that “specifically binds” to an antigen refers to an antigen-binding molecule or an antibody that binds to the antigen and substantially identical antigens with high affinity, which means having a KD of 10⁻⁷ M or less, preferably 10⁻⁸ M or less, even more preferably 10⁻⁹ M or less, and most preferably between 10⁻⁸ M and 10⁻¹⁰ M or less, but does not bind with high affinity to unrelated antigens, as measured by a surface plasmon resonance assay or a cell binding assay.

In some embodiments, binding activity or binding affinity (KD) of the antigen-binding domains of the present invention to the antigen of interest (i.e. PDGF-B or PDGF-D) are assessed at 25 degrees C. using e.g., Biacore T200 instrument (GE Healthcare). Anti-human Fc (e.g., GE Healthcare) is immobilized onto all flow cells of a CM4 sensor chip using amine coupling kit (e.g, GE Healthcare). The antigen-binding molecules or antigen-binding domains are captured onto the anti-Fc sensor surfaces, then the antigen (e.g. PDGF-B or PDGF-D) is injected over the flow cell. All antigen-binding domains and analytes are prepared in PBS-NET (10 mM phosphate, 287 mM NaCl, 2.7 mM KCl, 3.2 mM EDTA, 0.01% P20, 0.005% NaN₃, pH 7.4). Sensor surface is regenerated each cycle with 3M MgCl₂. Binding affinity are determined by processing and fitting the data to 1:1 binding model using e.g., Biacore T200 Evaluation software, version 2.0 (GE Healthcare).

### PDGF-B and PDGF-D

By the term “PDGF-B” is meant any naturally occurring form of PDGF-B, whether monomeric or dimeric, which may be derived from any suitable organisms. The term encompasses any dimer comprising PDGF-B, i.e., PDGF-AB and PDGF-BB. As used herein, “PDGF-B” refers to a mammalian PDGF-B, such as human, rat, or mouse, as well as non-human primate, bovine, ovine, or porcine PDGF-B. Preferably, the PDGF-B is human PDGF-B. The term “PDGF-B” also encompasses fragments, variants, isoforms, and other homologs of such PDGF-B molecules. Variant PDGF-B molecules will generally be characterized by having the same type of activity as naturally occurring PDGF-B, such as the ability to bind to PDGFR, the ability to induce phosphorylation of the receptor, the ability to mediate signaling by such receptor, the ability to induce cell migration or proliferation, and the ability to induce or increase deposition of extracellular matrix. The amino acid sequence of an exemplary human PDGF-B is shown in SEQ ID NO: 17.

Similarly, the term “PDGF-D” means any naturally occurring form of PDGF-D, whether monomeric or dimeric, which may be derived from any suitable organisms. The term encompasses dimer comprising PDGF-D, i.e., PDGF-DD. As used herein, “PDGF-D” refers to a mammalian PDGF-D, such as human, rat, or mouse, as well as non-human primate, bovine, ovine, or porcine PDGF-D. Preferably, the PDGF-D is human PDGF-D. The term “PDGF-D” also encompasses fragments, variants, isoforms, and other homologs of such PDGF-D molecules. Variant PDGF-D molecules will generally be characterized by having the same type of activity as naturally occurring PDGF-D, such as the ability to bind to PDGFR, the ability to induce phosphorylation of the receptor, the ability to mediate signaling by such receptor, the ability to induce cell migration or proliferation, and the ability to induce or increase deposition of extracellular matrix. The amino acid sequence of an exemplary human PDGF-D is shown in SEQ ID NO: 18.

In one aspect, the antigen-binding molecule of the present invention specifically binds to PDGF-B (e.g., PDGF-B, PDGF-AB, and PDGF-BB) and inhibits its interaction with PDGFR, thereby inhibiting PDGF-B activity. By the terms “PDGF-B mediated activity”, “PDGF-B mediated effect”, “PDGF-B activity”, “PDGF-B biological activity”, or “PDGF-B function”, as used interchangeably herein, is meant any activity mediated by PDGF-B interaction with a cognate receptor, the activity including, but not limited to, binding of PDGF-B to PDGFR, phosphorylation of PDGFR, increase in cell migration, increase in cell proliferation, increase in extracellular matrix deposition, and any other activity of PDGF-B either known in the art or to be elucidated in the future. In one embodiment, the antigen-binding molecule of the present invention is an antibody that specifically binds to PDGF-B. In one embodiment, the extent of binding of the antigen-binding molecule/antibody of the present invention to an unrelated, non-PDGF-B protein is less than about 10% of the binding of the antibody to PDGF-B as measured, e.g., by a radioimmunoassay (RIA). In one embodiment, said non-PDGF-B is PDGF-A, PDGF-C, or PDGF-D. In certain embodiments, an antibody that binds to PDGF-B has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M). In certain embodiments, an anti-PDGF-B antibody binds to an epitope of PDGF-B that is conserved among PDGF-B from different species.

In another aspect, the antigen-binding molecule of the present invention specifically binds to PDGF-D (e.g., PDGF-D and PDGF-DD) and inhibits its interaction with PDGFR, thereby inhibiting PDGF-D activity. By the terms “PDGF-D mediated activity”, “PDGF-D mediated effect”, “PDGF-D activity”, “PDGF-D biological activity”, or “PDGF-Dfunction”, as used interchangeably herein, is meant any activity mediated by PDGF-D interaction with a cognate receptor, the activity including, but not limited to, binding of PDGF-D to PDGFR, phosphorylation of PDGFR, increase in cell migration, increase in cell proliferation, increase in extracellular matrix deposition, and any other activity of PDGF-D either known in the art or to be elucidated in the future. In one embodiment, the antigen-binding molecule of the present invention is an antibody that specifically binds to PDGF-D. In one embodiment, the extent of binding of the antigen-binding molecule/antibody of the present invention to an unrelated, non-PDGF-D protein is less than about 10% of the binding of the antibody to PDGF-D as measured, e.g., by a radioimmunoassay (RIA). In one embodiment, said non-PDGF-D is PDGF-A, PDGF-B, or PDGF-C. In certain embodiments, an antibody that binds to PDGF-D has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M). In certain embodiments, an anti-PDGF-D antibody binds to an epitope of PDGF-D that is conserved among PDGF-D from different species.

In another aspect, the antigen-binding molecule of the present invention is a multispecific antigen-binding molecule, preferably a multispecific antibody, which specifically binds to PDGF-B (i.e., PDGF-B, PDGF-AB and PDGF-BB) and PDGF-D (i.e., PDGF-D and PDGF-DD) and inhibits their interaction with PDGFR, thereby inhibiting PDGF-B and PDGF-D activity.

Thus, the methods of the present invention use the antigen-binding molecule or antibody of the present invention that blocks, suppresses, or reduces (including significantly reduces) PDGF-B and/or PDGF-D activity, including downstream events mediated by PDGF-B and/or PDGF-D. An antigen-binding molecule or antibody of the present invention exhibits any one or more of the following characteristics: (a) specifically binding to PDGF-B and/or PDGF-D; (b) blocking PDGF-B and/or PDGF-D interaction with a cell surface receptor and downstream signaling events; (c) blocking phosphorylation of the PDGFR; (d) blocking PDGF-B and/or PDGF-D mediated induction of cell proliferation; (e) blocking PDGF-B and/or PDGF-D mediated induction of cell migration; and (f) blocking or reducing PDGF-B and/or PDGF-D mediated deposition of extracellular matrix. In one preferred embodiment, the antigen-binding molecule or antibody of the present invention preferably reacts with PDGF-B and/or PDGF-D in a manner that blocks PDGF-B and/or PDGF-D interaction with a cell surface receptor, e.g., PDGFR.

Affinity

“Affinity” refers to the strength of the sum total of noncovalent interactions between a single binding site of a molecule (e.g., an antigen-binding molecule or antibody) and its binding partner (e.g., an antigen). Unless indicated otherwise, as used herein, “binding affinity” refers to intrinsic binding affinity which reflects a 1:1 interaction between members of a binding pair (e.g., antigen-binding molecule and antigen, or antibody and antigen). The affinity of a molecule X for its partner Y can generally be represented by the dissociation constant (KD). Affinity can be measured by common methods known in the art, including those described herein. Specific illustrative and exemplary embodiments for measuring binding affinity are described in the following.

### Methods to Determine Affinity

In certain embodiments, the antigen-binding domain of an antigen-binding molecule or antibody provided herein has a dissociation constant (KD) of 1 micro M or less, 120 nM or less, 100 nM or less, 80 nM or less, 70 nM or less, 50 nM or less, 40 nM or less, 30 nM or less, 20 nM or less, 10 nM or less, 2 nM or less, 1 nM or less, 0.1 nM or less, 0.01 nM or less, or 0.001 nM or less (e.g., 10⁻⁸ M or less, 10⁻⁸ M to 10⁻¹³ M, 10⁻⁹M to 10⁻¹³ M) for its antigen. In certain embodiments, the KD value of the first antigen-binding domain of the antibody/antigen-binding molecule for PDGF-B or PDGF-D falls within the range of 1-40, 1-50, 1-70, 1-80, 30-50, 30-70, 30-80, 40-70, 40-80, or 60-80 nM.

In one embodiment, KD is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I] antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate is washed eight times with 0.1% polysorbate 20 (TWEEN-20 (registered trademark)) in PBS. When the plates have dried, 150 micro 1/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, KD is measured using a BIACORE (registered trademark) surface plasmon resonance assay. For example, an assay using a BIACORE (registered trademark)-2000 or a BIACORE (registered trademark)-3000 (BIAcore, Inc., Piscataway, N.J.) is performed at 25 degrees C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the suppliers instructions. Antigen is diluted with 10 mM sodium acetate, pH 4.8, to 5 micro g/ml (˜0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min Association rates (kon) and dissociation rates (koff) are calculated using a simple one-to-one Langmuir binding model (BIACORE (registered trademark) Evaluation Software version 3.2) by simultaneously fitting the association and dissociation sensorgrams. The equilibrium dissociation constant (KD) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

Methods for measuring the affinity of the antigen-binding domain of an antibody are described above, and one skilled in art can carry out affinity measurement for other antigen-binding domains.

### Antibody

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

### Class of Antibody

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

### Framework

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

### Human Consensus Framework

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

### HVR

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

HVR-H1, HVR-H2, HVR-H3, HVR-L1, HVR-L2, and HVR-L3 may also be called as “HCDR1”, “HCDR2”, “HCDR3”, LCDR1″, “LCDR2”, and “LCDR3”, respectively.

### Variable Region

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### Identity (Sequence Identity)

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Chimeric Antibody

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

### Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “humanized antibody variable region” refers to the variable region of a humanized antibody.

### Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human antibody variable region” refers to the variable region of a human antibody.

Methods for Producing an Antibody with Desired Binding Activity

Methods for producing an antibody with desired binding activity are known to those skilled in the art. Below is an example that describes a method for producing an antibody that binds to PDGF-B or PDGF-D (i.e. anti-PDGF-B antibody or anti-PDGF-D antibody).

Anti-PDGF-B or PDGF-D antibodies can be obtained as polyclonal or monoclonal antibodies using known methods. The anti-PDGF-B or PDGF-D antibodies produced are preferably monoclonal antibodies derived from mammals. Such mammal-derived monoclonal antibodies include antibodies produced by hybridomas or host cells transformed with an expression vector carrying an antibody gene by genetic engineering techniques.

Monoclonal antibody-producing hybridomas can be produced using known techniques, for example, as described below. Specifically, mammals are immunized by conventional immunization methods using a PDGF-B or PDGF-D protein as a sensitizing antigen. Resulting immune cells are fused with known parental cells by conventional cell fusion methods. Then, hybridomas producing an anti-PDGF-B or PDGF-D antibody can be selected by screening for monoclonal antibody-producing cells using conventional screening methods.

Specifically, monoclonal antibodies are prepared as mentioned below. First, a polypeptide comprising PDGF-B or PDGF-D, which will be used as a sensitizing antigen or immunogen for antibody preparation, can be synthesized or expressed. Alternatively, a nucleic acid encoding the full length PDGF-B or PDGF-D or truncated or other variant forms of PDGF-B or PDGF-D can be expressed to produce a PDGF-B or PDGF-D-containing protein (either in a monomeric form or in a dimeric form). The desired human full length PDGF-B or PDGF-D (monomer or dimer), truncated or other variant forms of PDGF-B or PDGF-D can be purified from the host cells or their culture supernatants by known methods.

The purified full length PDGF-B or PDGF-D, or truncated or other variant forms of PDGF-B or PDGF-D, can be used as a sensitizing antigen or immunogen for use in immunization of mammals. Partial peptides of full-length PDGF-B or PDGF-D can also be used as a sensitizing antigen. In this case, the partial peptides may also be obtained by chemical synthesis from the human PDGF-B or PDGF-D amino acid sequence. Furthermore, they may also be obtained by incorporating a portion of the PDGF-B or PDGF-D gene into an expression vector and expressing it.

Alternatively, it is possible to use a fusion protein prepared by fusing a desired partial polypeptide or peptide of the full-length PDGF-B or PDGF-D or a PDGF-B or PDGF-D ECD protein with a different polypeptide as a sensitizing antigen. For example, antibody Fc fragments and peptide tags are preferably used to produce fusion proteins to be used as sensitizing antigens. Vectors for expression of such fusion proteins can be constructed by fusing genes encoding two or more desired polypeptide fragments in-frame and inserting the fusion gene into an expression vector as described above. Methods for producing fusion proteins are described in Molecular Cloning 2nd ed. (Sambrook, J et al., Molecular Cloning 2nd ed., 9.47-9.58 (1989) Cold Spring Harbor Lab. Press). Methods for preparing PDGF-B or PDGF-D to be used as a sensitizing antigen, and immunization methods using PDGF-B or PDGF-D are also described in the Examples of this specification later.

There is no particular limitation on the mammals to be immunized with the sensitizing antigen. However, it is preferable to select the mammals by considering their compatibility with the parent cells to be used for cell fusion. In general, rodents such as mice, rats, and hamsters, rabbits, and monkeys are preferably used.

The above animals are immunized with a sensitizing antigen by known methods. Generally performed immunization methods include, for example, intraperitoneal or subcutaneous injection of a sensitizing antigen into mammals. Specifically, a sensitizing antigen is appropriately diluted with PBS (Phosphate-Buffered Saline), physiological saline, or the like. If desired, a conventional adjuvant such as Freund's complete adjuvant is mixed with the antigen, and the mixture is emulsified. Then, the sensitizing antigen is administered to a mammal several times at 4- to 21-day intervals. Appropriate carriers may be used in immunization with the sensitizing antigen. In particular, when a low-molecular-weight partial peptide is used as the sensitizing antigen, it is sometimes desirable to couple the sensitizing antigen peptide to a carrier protein such as albumin or keyhole limpet hemocyanin for immunization.

Alternatively, hybridomas producing a desired antibody can be prepared using DNA immunization as mentioned below. DNA immunization is an immunization method that confers immunostimulation by expressing a sensitizing antigen in an animal immunized as a result of administering a vector DNA constructed to allow expression of an antigen protein-encoding gene in the animal.

In order to prepare a monoclonal antibody of the present invention using DNA immunization, first, a DNA for the expression of a PDGF-B or PDGF-D protein is administered to an animal to be immunized. The PDGF-B or PDGF-D-encoding DNA can be synthesized by known methods such as PCR. The obtained DNA is inserted into an appropriate expression vector, and then this is administered to an animal to be immunized Preferably used expression vectors include, for example, commercially-available expression vectors such as pcDNA3.1. Vectors can be administered to an organism using conventional methods. For example, DNA immunization is performed by using a gene gun to introduce expression vector-coated gold particles into cells in the body of an animal to be immunized

After immunizing a mammal as described above, an increase in the titer of a PDGF-B or PDGF-D-binding antibody is confirmed in the serum. Then, immune cells are collected from the mammal, and then subjected to cell fusion. In particular, splenocytes are preferably used as immune cells.

A mammalian myeloma cell is used as a cell to be fused with the above-mentioned immunocyte. The myeloma cells preferably comprise a suitable selection marker for screening. A selection marker confers characteristics to cells for their survival (or death) under a specific culture condition. Hypoxanthine-guanine phosphoribosyltransferase deficiency (hereinafter abbreviated as HGPRT deficiency) and thymidine kinase deficiency (hereinafter abbreviated as TK deficiency) are known as selection markers. Cells with HGPRT or TK deficiency have hypoxanthine-aminopterin-thymidine sensitivity (hereinafter abbreviated as HAT sensitivity). HAT-sensitive cells cannot synthesize DNA in a HAT selection medium and thus are killed by culturing in the HAT selection medium; however, they are rescued if the cells are fused with normal cells. The cells can continue DNA synthesis using the salvage pathway of the normal cells, and therefore they can grow even in the HAT selection medium.

HGPRT-deficient cells can be selected in a medium containing 6-thioguanine or 8-azaguanine (hereinafter abbreviated as 8AG), and TK-deficient cells can be selected in a medium containing 5′-bromodeoxyuridine. Normal cells are killed in the selection medium because they incorporate these pyrimidine analogs into their DNA. Meanwhile, cells that are deficient in these enzymes can survive in the selection medium, since they cannot incorporate these pyrimidine analogs. In addition, a selection marker referred to as G418 resistance provided by the neomycin-resistant gene confers resistance to 2-deoxystreptamine antibiotics (gentamycin analogs). Various types of myeloma cells that are suitable for cell fusion are known.

For example, myeloma cells including the following cells can be preferably used:
P3(P3 x63Ag8.653) (J. Immunol. (1979) 123 (4), 1548-1550);
P3 x63Ag8U.1 (Current Topics in Microbiology and Immunology (1978) 81, 1-7);
NS-1 (C. Eur. J. Immunol. (1976) 6 (7), 511-519);
MPC-11 (Cell (1976) 8 (3), 405-415);
SP2/0 (Nature (1978) 276 (5685), 269-270);
FO (J. Immunol. Methods (1980) 35 (1-2), 1-21);
S194/5.XX0.BU.1 (J. Exp. Med. (1978) 148 (1), 313-323);
R210 (Nature (1979) 277 (5692), 131-133), etc.

Cell fusions between the immunocytes and myeloma cells are essentially carried out using known methods, for example, a method by Kohler and Milstein et al. (Methods Enzymol. (1981) 73: 3-46).

More specifically, cell fusion can be carried out, for example, in a conventional culture medium in the presence of a cell fusion-promoting agent. The fusion-promoting agents include, for example, polyethylene glycol (PEG) and Sendai virus (HVJ). If required, an auxiliary substance such as dimethyl sulfoxide is also added to improve fusion efficiency.

The ratio of immunocytes to myeloma cells may be determined suitably, and preferable example includes one myeloma cell for every one to ten immunocytes. Culture media to be used for cell fusion include, for example, media that are suitable for the growth of myeloma cell lines, such as RPMI1640 medium and MEM medium, and other conventional culture medium used for this type of cell culture. In addition, serum supplements such as fetal calf serum (FCS) may be preferably added to the culture medium.

For cell fusion, predetermined number of the above immune cells and myeloma cells are mixed well in the above culture medium. Then, a PEG solution (for example, the average molecular weight is about 1,000 to 6,000) prewarmed to about 37 degrees C. is added thereto at a concentration of generally 30% to 60% (w/v). This is gently mixed to produce desired fusion cells (hybridomas). Then, an appropriate culture medium mentioned above is gradually added to the cells, and this is repeatedly centrifuged to remove the supernatant. Thus, cell fusion agents and such which are unfavorable to hybridoma growth can be removed.

The hybridomas thus obtained can be selected by culturing using a conventional selective medium, for example, HAT medium (a culture medium containing hypoxanthine, aminopterin, and thymidine). Cells other than the desired hybridomas (non-fused cells) can be killed by continuing culturing in the above HAT medium for a sufficient period of time. Typically, the period is several days to several weeks. Then, hybridomas producing the desired antibody are screened for and singly cloned by conventional limiting dilution methods.

The hybridomas thus obtained can be selected using a selection medium based on the selection marker possessed by the myeloma used for cell fusion. For example, HGPRT- or TK-deficient cells can be selected by culturing using the HAT medium (a culture medium containing hypoxanthine, aminopterin, and thymidine). Specifically, when HAT-sensitive myeloma cells are used for cell fusion, cells successfully fused with normal cells can selectively proliferate in the HAT medium. Cells other than the desired hybridomas (non-fused cells) can be killed by continuing culturing in the above HAT medium for a sufficient period of time. Specifically, desired hybridomas can be selected by culturing for generally several days to several weeks. Then, hybridomas producing the desired antibody are screened for and singly cloned by conventional limiting dilution methods.

Monoclonal antibody-producing hybridomas thus prepared can be passaged in a conventional culture medium and stored in liquid nitrogen for a long period of time.

The above hybridomas are cultured by a conventional method, and desired monoclonal antibodies can be prepared from the culture supernatants. Alternatively, the hybridomas are administered to and grown in compatible mammals, and monoclonal antibodies are prepared from the ascites. The former method is suitable for preparing antibodies with high purity.

Antibodies encoded by antibody genes that are cloned from antibody-producing cells such as the above hybridomas can also be preferably used. A cloned antibody gene is inserted into an appropriate vector, and this is introduced into a host to express the antibody encoded by the gene. Methods for isolating antibody genes, inserting the genes into vectors, and transforming host cells have already been established, for example, by Vandamme et al. (Eur. J. Biochem. (1990) 192(3), 767-775). Methods for producing recombinant antibodies are also known as described below.

Preferably, the present invention provides nucleic acids that encode an antigen-binding molecule or a multispecific antigen-binding molecule of the present invention. The present invention also provides a vector into which the nucleic acid encoding the antigen-binding molecule or multispecific antigen-binding molecule is introduced, i.e., a vector comprising the nucleic acid. Furthermore, the present invention provides a cell comprising the nucleic acid or the vector. The present invention also provides a method for producing the antigen-binding molecule or multispecific antigen-binding molecule by culturing the cell. The present invention further provides antigen-binding molecules or multispecific antigen-binding molecules produced by the method.

For example, a cDNA encoding the variable region (V region) of an anti-PDGF-B or PDGF-D antibody is prepared from hybridoma cells expressing the anti-PDGF-B or PDGF-D antibody. For this purpose, total RNA is first extracted from hybridomas. Methods used for extracting mRNAs from cells include, for example:
the guanidine ultracentrifugation method (Biochemistry (1979) 18(24), 5294-5299), and
the AGPC method (Anal. Biochem. (1987) 162(1), 156-159).

Extracted mRNAs can be purified using the mRNA Purification Kit (GE Healthcare Bioscience) or such. Alternatively, kits for extracting total mRNA directly from cells, such as the QuickPrep mRNA Purification Kit (GE Healthcare Bioscience), are also commercially available. mRNAs can be prepared from hybridomas using such kits. cDNAs encoding the antibody V region can be synthesized from the prepared mRNAs using a reverse transcriptase. cDNAs can be synthesized using the AMV Reverse Transcriptase First-strand cDNA Synthesis Kit (Seikagaku Co.) or such. Furthermore, the SMART RACE cDNA amplification kit (Clontech) and the PCR-based 5′-RACE method (Proc. Natl. Acad. Sci. USA (1988) 85(23), 8998-9002; Nucleic Acids Res. (1989) 17(8), 2919-2932) can be appropriately used to synthesize and amplify cDNAs. In such a cDNA synthesis process, appropriate restriction enzyme sites described below may be introduced into both ends of a cDNA.

The cDNA fragment of interest is purified from the resulting PCR product, and then this is ligated to a vector DNA. A recombinant vector is thus constructed and introduced into E. coli or such. After colony selection, the desired recombinant vector can be prepared from the colony-forming E. coli. Then, whether the recombinant vector has the cDNA nucleotide sequence of interest is tested by a known method such as the dideoxy nucleotide chain termination method.

The 5′-RACE method which uses primers to amplify the variable region gene is conveniently used for isolating the gene encoding the variable region. First, a 5′-RACE cDNA library is constructed by cDNA synthesis using RNAs extracted from hybridoma cells as a template. A commercially available kit such as the SMART RACE cDNA amplification kit is appropriately used to synthesize the 5′-RACE cDNA library.

The antibody gene is amplified by PCR using the prepared 5′-RACE cDNA library as a template. Primers for amplifying the mouse antibody gene can be designed based on known antibody gene sequences. The nucleotide sequences of the primers vary depending on the immunoglobulin subclass. Therefore, it is preferable that the subclass is determined in advance using a commercially available kit such as the Iso Strip mouse monoclonal antibody isotyping kit (Roche Diagnostics).

Specifically, for example, primers that allow amplification of genes encoding gamma1, gamma2a, gamma2b, and gamma3 heavy chains and kappa and lambda light chains are used to isolate mouse IgG-encoding genes. In general, a primer that anneals to a constant region site close to the variable region is used as a 3′-side primer to amplify an IgG variable region gene. Meanwhile, a primer attached to a 5′ RACE cDNA library construction kit is used as a 5′-side primer.

PCR products thus amplified are used to reshape immunoglobulins composed of a combination of heavy and light chains. A desired antibody can be selected using the PDGF-B or PDGF-D-binding activity of a reshaped immunoglobulin as an indicator. For example, when the objective is to isolate an antibody against PDGF-B or PDGF-D, it is more preferred that the binding of the antibody to PDGF-B or PDGF-D is specific. A PDGF-B or PDGF-D-binding antibody can be screened for, for example, by the following steps:
(1) contacting a PDGF-B or PDGF-D with an antibody comprising the V region encoded by a cDNA isolated from a hybridoma;
(2) detecting the binding of the antibody to the PDGF-B or PDGF-D;
(3) selecting an antibody that specifically binds to the PDGF-B or PDGF-D;

and preferably
(4) selecting an antibody that shows strong binding to PDGF-B or PDGF-D.

Preferred antibody screening methods that use the binding activity as an indicator also include panning methods using phage vectors. Screening methods using phage vectors are advantageous when the antibody genes are isolated from heavy-chain and light-chain subclass libraries from a polyclonal antibody-expressing cell population. Genes encoding the heavy-chain and light-chain variable regions can be linked by an appropriate linker sequence to form a single-chain Fv (scFv). Phages presenting scFv on their surface can be produced by inserting a gene encoding scFv into a phage vector. The phages are contacted with an antigen of interest. Then, a DNA encoding scFv having the binding activity of interest can be isolated by collecting phages bound to the antigen. This process can be repeated as necessary to enrich scFv having a desired binding activity.

After the isolation of the cDNA encoding the V region of the anti-PDGF-B or PDGF-D antibody of interest, the cDNA is digested with restriction enzymes that recognize the restriction sites introduced into both ends of the cDNA. Preferred restriction enzymes recognize and cleave a nucleotide sequence that occurs in the nucleotide sequence of the antibody gene at a low frequency. Furthermore, a restriction site for an enzyme that produces a sticky end is preferably introduced into a vector to insert a single-copy digested fragment in the correct orientation. The cDNA encoding the V region of the anti-PDGF-B or PDGF-D antibody is digested as described above, and this is inserted into an appropriate expression vector to construct an antibody expression vector. In this case, if a gene encoding the antibody constant region (C region) and a gene encoding the above V region are fused in-frame, a chimeric antibody is obtained. Herein, “chimeric antibody” means that the origin of the constant region is different from that of the variable region. Thus, in addition to mouse/human heterochimeric antibodies, human/human allochimeric antibodies are included in the chimeric antibodies of the present invention. A chimeric antibody expression vector can be constructed by inserting the above V region gene into an expression vector that already has the constant region. Specifically, for example, a recognition sequence for a restriction enzyme that excises the above V region gene can be appropriately placed on the 5′ side of an expression vector carrying a DNA encoding a desired antibody constant region (C region). A chimeric antibody expression vector is constructed by fusing in-frame the two genes digested with the same combination of restriction enzymes.

To produce an anti-PDGF-B or PDGF-D monoclonal antibody, antibody genes are inserted into an expression vector so that the genes are expressed under the control of an expression regulatory region. The expression regulatory region for antibody expression includes, for example, enhancers and promoters. Furthermore, an appropriate signal sequence may be attached to the amino terminus so that the expressed antibody is secreted to the outside of cells. Meanwhile, other appropriate signal sequences may be attached. The expressed polypeptide is cleaved at the carboxyl terminus of the above sequence, and the resulting polypeptide is secreted to the outside of cells as a mature polypeptide. Then, appropriate host cells are transformed with the expression vector, and recombinant cells expressing the anti-PDGF-B or PDGF-D antibody-encoding DNA are obtained.

DNAs encoding the antibody heavy chain (H chain) and light chain (L chain) are separately inserted into different expression vectors to express the antibody gene. An antibody molecule having the H and L chains can be expressed by co-transfecting the same host cell with vectors into which the H-chain and L-chain genes are respectively inserted. Alternatively, host cells can be transformed with a single expression vector into which DNAs encoding the H and L chains are inserted (see WO 94/11523).

There are various known host cell/expression vector combinations for antibody preparation by introducing isolated antibody genes into appropriate hosts. These expression systems are all applicable to isolation of domains including antibody variable regions of the present invention. Appropriate eukaryotic cells used as host cells include animal cells, plant cells, and fungal cells. Specifically, the animal cells include, for example, the following cells.
(1) mammalian cells: CHO, COS, myeloma, baby hamster kidney (BHK), HeLa,

Vero, or such;
(2) amphibian cells: Xenopus oocytes, or such; and
(3) insect cells: sf9, sf21, Tn5, or such.

In addition, as a plant cell, an antibody gene expression system using cells derived from the Nicotiana genus such as Nicotiana tabacum is known. Callus cultured cells can be appropriately used to transform plant cells.

Furthermore, the following cells can be used as fungal cells: yeasts: the Saccharomyces genus such as Saccharomyces cerevisiae, and the Pichia genus such as Pichia pastoris; and filamentous fungi: the Aspergillus genus such as Aspergillus niger.

Furthermore, antibody gene expression systems that utilize prokaryotic cells are also known. For example, when using bacterial cells, E. coli cells, Bacillus subtilis cells, and such can suitably be utilized in the present invention. Expression vectors carrying the antibody genes of interest are introduced into these cells by transfection. The transfected cells are cultured in vitro, and the desired antibody can be prepared from the culture of the transformed cells.

In addition to the above-described host cells, transgenic animals can also be used to produce a recombinant antibody. That is, the antibody can be obtained from an animal into which the gene encoding the antibody of interest is introduced. For example, the antibody gene can be constructed as a fusion gene by inserting it in-frame into a gene that encodes a protein produced and secreted specifically into milk. Goat beta-casein or such can be used, for example, as the protein secreted to milk. DNA fragments containing the fusion gene comprising the introduced antibody gene is injected into a goat embryo, and then this embryo is introduced into a female goat. Desired antibodies can be obtained as a protein fused with the milk protein from milk produced by the transgenic goat born from the embryo-recipient goat (or progeny thereof). In addition, to increase the volume of milk containing the desired antibody produced by the transgenic goat, hormones can be administered to the transgenic goat as necessary (Ebert, K. M. et al., Bio/Technology (1994) 12 (7), 699-702).

### Methods for Producing a Humanized Antibody

When an antigen-binding molecule described herein is administered to human, a domain derived from a genetically recombinant antibody that has been artificially modified to reduce the heterologous antigenicity against human and such, can be appropriately used as the domain of the antigen-binding molecule including an antibody variable region. Such genetically recombinant antibodies include, for example, humanized antibodies. These modified antibodies are appropriately produced by known methods. Furthermore, generally, the binding specificity of a certain antibody can be introduced into another antibody by CDR grafting.

Specifically, humanized antibodies prepared by grafting the CDR of a non-human animal antibody such as a mouse antibody to a human antibody are known. Genetic engineering techniques for obtaining such humanized antibodies are also commonly known. Specifically, for example, overlap extension PCR is known as a method for grafting a mouse antibody CDR to a human FR. In overlap extension PCR, a nucleotide sequence encoding a mouse antibody CDR to be grafted is added to primers for synthesizing a human antibody FR. Primers are prepared for each of the four FRs. It is generally considered that when grafting a mouse CDR to a human FR, selecting a human FR that has high identity to a mouse FR is advantageous for maintaining the CDR function. That is, it is generally preferable to use a human FR comprising an amino acid sequence which has high identity to the amino acid sequence of the FR adjacent to the mouse CDR to be grafted.

Nucleotide sequences to be ligated are designed so that they will be connected to each other in-frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, complementary strand synthesis reaction is conducted to anneal the overlapping CDR regions of the products synthesized using a human antibody gene as template. Human FRs are ligated via the mouse CDR sequences by this reaction.

The full-length V region gene, in which three CDRs and four FRs are ligated, is amplified using primers that anneal to its 5′- or 3′-end, which are added with suitable restriction enzyme recognition sequences. An expression vector for humanized antibody can be produced by inserting the DNA obtained as described above and a DNA that encodes a human antibody C region into an expression vector so that they will ligate in-frame. After the recombinant vector is transfected into a host to establish recombinant cells, the recombinant cells are cultured, and the DNA encoding the humanized antibody is expressed to produce the humanized antibody in the cell culture (see, European Patent Publication No. EP 239400 and International Patent Publication No. WO 1996/002576).

By qualitatively or quantitatively measuring and evaluating the antigen-binding activity of the humanized antibody produced as described above, one can suitably select human antibody FRs that allow CDRs to form a favorable antigen-binding site when ligated through the CDRs Amino acid residues in FRs may be substituted as necessary, so that the CDRs of a reshaped human antibody form an appropriate antigen-binding site. For example, amino acid sequence mutations can be introduced into FRs by applying the PCR method used for grafting a mouse CDR into a human FR. More specifically, partial nucleotide sequence mutations can be introduced into primers that anneal to the FR. Nucleotide sequence mutations are introduced into the FRs synthesized by using such primers. Mutant FR sequences having the desired characteristics can be selected by measuring and evaluating the activity of the amino acid-substituted mutant antibody to bind to the antigen by the above-mentioned method (Sato, K. et al., Cancer Res. (1993) 53: 851-856)

### Methods for Producing a Human Antibody.

Alternatively, desired human antibodies can be obtained by immunizing transgenic animals having the entire repertoire of human antibody genes (see WO 1993/012227; WO 1992/003918; WO 1994/002602; WO 1994/025585; WO 1996/034096; WO 1996/033735) by DNA immunization.

Furthermore, techniques for preparing human antibodies by panning using human antibody libraries are also known. For example, the V region of a human antibody is expressed as a single-chain antibody (scFv) on phage surface by the phage display method. Phages expressing a scFv that binds to the antigen can be selected. The DNA sequence encoding the human antibody V region that binds to the antigen can be determined by analyzing the genes of selected phages. The DNA sequence of the scFv that binds to the antigen is determined. An expression vector is prepared by fusing the V region sequence in-frame with the C region sequence of a desired human antibody, and inserting this into an appropriate expression vector. The expression vector is introduced into cells appropriate for the expression such as those described above. The human antibody can be produced by expressing the human antibody-encoding gene in the cells. These methods are already known (see WO 1992/001047; WO 1992/020791; WO 1993/006213; WO 1993/011236; WO 1993/019172; WO 1995/001438; WO 1995/015388).

### Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### Host Cell

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to their parent cell, and may contain mutations. Mutant progeny that have the same function or biological activity as screened or selected for in the originally transformed cell are included herein.

### Epitope

“Epitope” means an antigenic determinant in an antigen, and refers to an antigen site to which the antigen-binding domain of an antigen-binding molecule or antibody disclosed herein binds. Thus, for example, the epitope can be defined according to its structure. Alternatively, the epitope may be defined according to the antigen-binding activity of an antigen-binding molecule or antibody that recognizes the epitope. When the antigen is a peptide or polypeptide, the epitope can be specified by the amino acid residues forming the epitope. Alternatively, when the epitope is a sugar chain, the epitope can be specified by its specific sugar chain structure.

A linear epitope is an epitope that contains an epitope whose primary amino acid sequence is recognized. Such a linear epitope typically contains at least three and most commonly at least five, for example, about 8 to 10 or 6 to 20 amino acids in its specific sequence.

In contrast to the linear epitope, “conformational epitope” is an epitope in which the primary amino acid sequence containing the epitope is not the only determinant of the recognized epitope (for example, the primary amino acid sequence of a conformational epitope is not necessarily recognized by an epitope-defining antibody). Conformational epitopes may contain a greater number of amino acids compared to linear epitopes. A conformational epitope-recognizing antigen-binding domain recognizes the three-dimensional structure of a peptide or protein. For example, when a protein molecule folds and forms a three-dimensional structure, amino acids and/or polypeptide main chains that form a conformational epitope become aligned, and the epitope is made recognizable by the antigen-binding domain. Methods for determining epitope conformations include, for example, X ray crystallography, two-dimensional nuclear magnetic resonance, site-specific spin labeling, and electron paramagnetic resonance, but are not limited thereto. See, for example, Epitope Mapping Protocols in Methods in Molecular Biology (1996), Vol. 66, Morris (ed.).

Examples of a method for assessing the epitope binding by a test antigen-binding molecule or antibody containing an anti-PDGF-B or PDGF-D antigen-binding domain are described below. According to the examples below, methods for assessing the epitope binding by a test antigen-binding molecule or antibody containing an antigen-binding domain for an antigen other than PDGF-B or PDGF-D, can also be appropriately conducted.

For example, whether a test antigen-binding molecule or antibody containing an anti-PDGF-B or PDGF-D antigen-binding domain recognizes a linear epitope in the PDGF-B or PDGF-D molecule can be confirmed for example as mentioned below. A linear peptide comprising an amino acid sequence of PDGF-B or PDGF-D is synthesized for the above purpose. The peptide can be synthesized chemically or obtained by genetic engineering techniques using a region of a PDGF-B or PDGF-D cDNA encoding the amino acid sequence. Then, a test antigen-binding molecule or antibody containing an anti-PDGF-B or PDGF-D antigen-binding domain is assessed for its binding activity towards a linear peptide comprising the amino acid sequence. For example, an immobilized linear peptide can be used as an antigen in ELISA to evaluate the binding activity of the polypeptide complex towards the peptide. Alternatively, the binding activity towards a linear peptide can be assessed based on the level that the linear peptide inhibits the binding of the antigen-binding molecule or antibody to PDGF-B or PDGF-D. These tests can demonstrate the binding activity of the antigen-binding molecule or antibody towards the linear peptide.

Whether a test antigen-binding molecule or antibody containing an anti-PDGF-B or PDGF-D antigen-binding domain recognizes a conformational epitope can be assessed as follows. A test antigen-binding molecule or antibody containing an anti-PDGF-B or PDGF-D antigen-binding domain can be determined to recognize a conformational epitope when it strongly binds to PDGF-B or PDGF-D upon contact, but does not substantially bind to an immobilized linear peptide comprising an amino acid sequence of PDGF-B or PDGF-D. Herein, “not substantially bind” means that the binding activity is 80% or less, generally 50% or less, preferably 30% or less, and particularly preferably 15% or less compared to the binding activity towards PDGF-B or PDGF-D.

Methods for assaying the binding activity of a test antigen-binding molecule or antibody containing an anti-PDGF-B or PDGF-D antigen-binding domain towards PDGF-B or PDGF-D include, for example, the methods described in Antibodies: A Laboratory Manual (Ed Harlow, David Lane, Cold Spring Harbor Laboratory (1988) 359-420).

In the ELISA format, the binding activity of a test antigen-binding molecule or antibody containing an anti-PDGF-B or PDGF-D antigen-binding domain towards PDGF-B or PDGF-D can be assessed quantitatively by comparing the levels of signal generated by enzymatic reaction. Specifically, a test antigen-binding molecule or antibody containing an anti-PDGF-B or PDGF-D antigen-binding domain is added to an ELISA plate onto which PDGF-B or PDGF-D are immobilized. Then, the test antigen-binding molecule or antibody bound to the PDGF-B or PDGF-D is detected using an enzyme-labeled antibody that recognizes the test antigen-binding molecule or antibody. Alternatively, when FACS is used, a dilution series of a test antigen-binding molecule or antibody is prepared, and the antibody binding titer for PDGF-B or PDGF-D can be determined to compare the binding activity of the test antigen-binding molecule or antibody towards PDGF-B or PDGF-D.

Whether a test antigen-binding molecule or antibody containing an anti-PDGF-B or PDGF-D antigen-binding domain shares a common epitope with another antigen-binding molecule or antibody can be assessed based on the competition between the two antigen-binding molecules or antibodies for the same epitope. The competition between the antigen-binding molecules or antibodies can be detected by cross-blocking assay or the like. For example, the competitive ELISA assay is a preferred cross-blocking assay.

Specifically, in cross-blocking assay, the PDGF-B or PDGF-D protein immobilized to the wells of a microtiter plate is pre-incubated in the presence or absence of a candidate competitor antigen-binding molecule or antibody, and then a test antigen-binding molecule or antibody is added thereto. The quantity of test antigen-binding molecule or antibody bound to the PDGF-B or PDGF-D protein in the wells is indirectly correlated with the binding ability of a candidate competitor antigen-binding molecule or antibody that competes for the binding to the same epitope. That is, the greater the affinity of the competitor antigen-binding molecule or antibody for the same epitope, the lower the binding activity of the test antigen-binding molecule or antibody towards the PDGF-B or PDGF-D protein-coated wells.

The quantity of the test antigen-binding molecule or antibody bound to the wells via the PDGF-B or PDGF-D protein can be readily determined by labeling the antigen-binding molecule or antibody in advance. For example, a biotin-labeled antigen-binding molecule or antibody is measured using an avidin/peroxidase conjugate and appropriate substrate. In particular, cross-blocking assay that uses enzyme labels such as peroxidase is called “competitive ELISA assay”. The antigen-binding molecule or antibody can also be labeled with other labeling substances that enable detection or measurement. Specifically, radiolabels, fluorescent labels, and such are known.

When the candidate competitor antigen-binding molecule or antibody can block the binding by a test antigen-binding molecule or antibody containing an anti-PDGF-B or PDGF-D antigen-binding domain by at least 20%, preferably at least 20 to 50%, and more preferably at least 50% compared to the binding activity in a control experiment conducted in the absence of the competitor antigen-binding molecule or antibody, the test antigen-binding molecule or antibody is determined to substantially bind to the same epitope bound by the competitor antigen-binding molecule or antibody, or compete for the binding to the same epitope.

When the structure of an epitope bound by a test antigen-binding molecule or antibody containing an anti-PDGF-B or PDGF-D antigen-binding domain has already been identified, whether the test and control antigen-binding molecules or antibodies share a common epitope can be assessed by comparing the binding activities of the two antigen-binding molecules or antibodies towards a peptide prepared by introducing amino acid mutations into the peptide forming the epitope.

To measure the above binding activities, for example, the binding activities of test and control antigen-binding molecules or antibodies towards a linear peptide into which a mutation is introduced are compared in the above ELISA format. Besides the ELISA methods, the binding activity towards the mutant peptide bound to a column can be determined by flowing test and control antigen-binding molecules or antibodies in the column, and then quantifying the antigen-binding molecule or antibody eluted in the elution solution. Methods for adsorbing a mutant peptide to a column, for example, in the form of a GST fusion peptide, are known.

### Specificity

“Specific” means that a molecule that binds specifically to one or more binding partners does not show any significant binding to molecules other than the partners. Furthermore, “specific” is also used when an antigen-binding domain is specific to a particular epitope of multiple epitopes contained in an antigen. When an epitope bound by an antigen-binding domain is contained in multiple different antigens, an antigen-binding molecule containing the antigen-binding domain can bind to various antigens that have the epitope.

### Monospecific Antigen-Binding Molecules

The term “monospecific antigen-binding molecule” is used to refer to antigen-binding molecules that specifically bind to only one type of antigen. A favorable example of a monospecific antigen-binding molecule is an antigen-binding molecule that comprises a single type of antigen-binding domain. Monospecific antigen-binding molecules can comprise a single antigen-binding domain or a plurality of antigen-binding domains of the same type. A favorable example of monospecific antigen-binding molecules is a monospecific antibody. When the monospecific antigen-binding molecule is a monospecific antibody of the IgG form, the monospecific antibody comprises two antibody variable fragments that have the same antigen-binding specificity.

### Antibody Fragment

An “antibody fragment” refers to a molecule that is other than an intact antibody but comprises a portion of the intact antibody and binds to the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The terms “full length antibody”, “intact antibody”, and “whole antibody” are used herein interchangeably to refer to an antibody having a structure substantially similar to a native antibody structure or having heavy chains that contain an Fc region as defined herein.

### Variable Fragment (Fv)

Herein, the term “variable fragment (Fv)” refers to the minimum unit of an antibody-derived antigen-binding domain that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

scFv, Single-Chain Antibody, and Sc(Fv)₂

Herein, the terms “scFv”, “single-chain antibody”, and “sc(Fv)₂” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized

scFv is an antigen-binding domain in which VH and VL forming Fv are linked together by a peptide linker (Proc. Natl. Acad. Sci. U.S.A. (1988) 85(16), 5879-5883). VH and VL can be retained in close proximity by the peptide linker.

sc(Fv)₂ is a single-chain antibody in which four variable regions, i.e. two VL and two VH, are linked by linkers such as peptide linkers to form a single chain (J Immunol. Methods (1999) 231(1-2), 177-189). The two VH and two VL may be derived from different monoclonal antibodies. Such sc(Fv)₂ preferably includes, for example, a bispecific sc(Fv)₂ that recognizes two epitopes present in a single antigen as disclosed in the Journal of Immunology (1994) 152(11), 5368-5374. sc(Fv)₂ can be produced by methods known to those skilled in the art. For example, sc(Fv)₂ can be produced by linking scFv by a linker such as a peptide linker.

Herein, the form of an antigen-binding domain forming an sc(Fv)₂ include an antibody in which the two VH units and two VL units are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Examples of the form are listed below.

[VL]-linker-[VH]-linker-[VH]-linker-[VL] [VH]-linker-[VL]-linker-[VL]-linker-[VH] [VH]-linker-[VH]-linker-[VL]-linker-[VL] [VL]-linker-[VL]-linker-[VH]-linker-[VH] [VL]-linker-[VH]-linker-[VL]-linker-[VH]

The molecular form of sc(Fv)₂ is also described in detail in WO 2006/132352. According to these descriptions, those skilled in the art can appropriately prepare desired sc(Fv)₂ to produce the polypeptide complexes disclosed herein.

Furthermore, the antigen-binding molecules or antibodies of the present invention may be conjugated with a carrier polymer such as PEG or an organic compound such as an anticancer agent. Alternatively, a sugar chain addition sequence is preferably inserted into the antigen-binding molecules or antibodies such that the sugar chain produces a desired effect.

The linkers to be used for linking the variable regions of an antibody comprise arbitrary peptide linkers that can be introduced by genetic engineering, synthetic linkers, and linkers disclosed, for example, in Protein Engineering, 9(3), 299-305, 1996. However, peptide linkers are preferred in the present invention. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. When sc(Fv)₂ contains three peptide linkers, their length may be all the same or different.

For example, such peptide linkers include:

Ser
Gly Ser
Gly Gly Ser
Ser Gly Gly
(SEQ ID NO: 33)
Gly Gly Gly Ser
(SEQ ID NO: 34)
Ser Gly Gly Gly
(SEQ ID NO: 35)
Gly Gly Gly Gly Ser
(SEQ ID NO: 36)
Ser Gly Gly Gly Gly
(SEQ ID NO: 37)
Gly Gly Gly Gly Gly Ser
(SEQ ID NO: 38)
Ser Gly Gly Gly Gly Gly
(SEQ ID NO: 39)
Gly Gly Gly Gly Gly Gly Ser
(SEQ ID NO: 40)
Ser Gly Gly Gly Gly Gly Gly
(Gly Gly Gly Gly Ser (SEQ ID NO: 35))n
(Ser Gly Gly Gly Gly (SEQ ID NO: 36))n

where n is an integer of 1 or larger. The length or sequences of peptide linkers can be selected accordingly by those skilled in the art depending on the purpose.

Synthetic linkers (chemical crosslinking agents) are routinely used to crosslink peptides, and examples include:
N-hydroxy succinimide (NHS),
disuccinimidyl suberate (DSS),
bis(sulfosuccinimidyl) suberate (BS3),
dithiobis(succinimidyl propionate) (DSP),
dithiobis(sulfosuccinimidyl propionate) (DTSSP),
ethylene glycol bis(succinimidyl succinate) (EGS),
ethylene glycol bis(sulfosuccinimidyl succinate) (sulfo-EGS),
disuccinimidyl tartrate (DST), disulfosuccinimidyl tartrate (sulfo-DST),
bis[2-(succinimidoxycarbonyloxy)ethyl] sulfone (BSOCOES), and
bis[2-(sulfosuccinimidoxycarbonyloxy)ethyl] sulfone (sulfo-BSOCOES).
These crosslinking agents are commercially available.

In general, three linkers are required to link four antibody variable regions together. The linkers to be used may be of the same type or different types.

### Fab, F(ab′)₂, and Fab′

“Fab” consists of a single light chain, and a CH1 domain and variable region from a single heavy chain. The heavy chain of Fab molecule cannot form disulfide bonds with another heavy chain molecule.

“F(ab′)₂” or “Fab” is produced by treating an immunoglobulin (monoclonal antibody) with a protease such as pepsin and papain, and refers to an antibody fragment generated by digesting an immunoglobulin (monoclonal antibody) near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)₂” consists of two light chains and two heavy chains comprising the constant region of a CH1 domain and a portion of CH2 domains so that disulfide bonds are formed between the two heavy chains. The F(ab′)₂ disclosed herein can be preferably produced as follows. A whole monoclonal antibody or such comprising a desired antigen-binding domain is partially digested with a protease such as pepsin; and Fc fragments are removed by adsorption onto a Protein A column. The protease is not particularly limited, as long as it can cleave the whole antibody in a selective manner to produce F(ab′)₂ under an appropriate setup enzyme reaction condition such as pH. Such proteases include, for example, pepsin and ficin.

### Fc Region

The term “Fc region” or “Fc domain” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

### Fc Receptor

The term “Fc receptor” or “FcR” refers to a receptor that binds to the Fc region of an antibody. In some embodiments, an FcR is a native human FcR. In some embodiments, an FcR is one which binds to an IgG antibody (a gamma receptor) and includes receptors of the Fc gamma RI, Fc gamma RII, and Fc gamma RIB subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

### Fc Gamma Receptor

Fc gamma receptor refers to a receptor capable of binding to the Fc domain of monoclonal IgG1, IgG2, IgG3, or IgG4 antibodies, and includes all members belonging to the family of proteins substantially encoded by an Fc gamma receptor gene. In human, the family includes Fc gamma RI (CD64) including isoforms Fc gamma RIa, Fc gamma RIb and Fc gamma RIc; Fc gamma RII (CD32) including isoforms Fc gamma RIIa (including allotype H131 and R131), Fc gamma RIIb (including Fc gamma RIIb-1 and Fc gamma RIIb-2), and Fc gamma RIIc; and Fc gamma RIII (CD16) including isoform Fc gamma RIIIa (including allotype V158 and F158) and Fc gamma RIIIb (including allotype Fc gamma RIIIb-NA1 and Fc gamma RIIIb-NA2); as well as all unidentified human Fc gamma receptors, Fc gamma receptor isoforms, and allotypes thereof. However, Fc gamma receptor is not limited to these examples. Without being limited thereto, Fc gamma receptor includes those derived from humans, mice, rats, rabbits, and monkeys. Fc gamma receptor may be derived from any organisms. Mouse Fc gamma receptor includes, without being limited to, Fc gamma RI (CD64), Fc gamma RII (CD32), Fc gamma RIII (CD16), and Fc gamma RIII-2 (CD16-2), as well as all unidentified mouse Fc gamma receptors, Fc gamma receptor isoforms, and allotypes thereof. Such preferred Fc gamma receptors include, for example, human Fc gamma RI (CD64), Fc gamma RIIA (CD32), Fc gamma RIIB (CD32), Fc gamma RIIIA (CD16), and/or Fc gamma RIIIB (CD16). The polynucleotide sequence and amino acid sequence of Fc gamma RI are shown in SEQ ID NOs: 28 (NM_000566.3) and 23 (NP_000557.1), respectively; the polynucleotide sequence and amino acid sequence of Fc gamma RIIA are shown in SEQ ID NOs: 29 (BCO20823.1) and 24 (AAH20823.1), respectively; the polynucleotide sequence and amino acid sequence of Fc gamma RIIB are shown in SEQ ID NOs: 30 (BC146678.1) and 25 (AAI46679.1), respectively; the polynucleotide sequence and amino acid sequence of Fc gamma RIIIA are shown in SEQ ID NOs: 31 (BC033678.1) and 26 (AAH33678.1), respectively; and the polynucleotide sequence and amino acid sequence of Fc gamma RIIIB are shown in SEQ ID NOs: 32 (BC128562.1) and 27 (AAI28563.1), respectively (RefSeq accession number is shown in each parentheses). Whether an Fc gamma receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE method, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, Fc gamma receptor, Fc alpha receptor, Fc beta receptor, FcRn, Clq, and C3, mannan-binding lectin, mannose receptor, Staphylococcus Protein A, Staphylococcus Protein G, and viral Fc gamma receptors. The Fc ligands also include Fc receptor homologs (FcRH) (Davis et al., (2002) Immunological Reviews 190, 123-136), which are a family of Fc receptors homologous to Fc gamma receptor. The Fc ligands also include unidentified molecules that bind to Fc.

### Fc Gamma Receptor-Binding Activity

The impaired binding activity of Fc domain to any of the Fc gamma receptors Fc gamma RI, Fc gamma RIIA, Fc gamma RIIB, Fc gamma RIIIA, and/or Fc gamma RIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103(11), 4005-4010).

ALPHA screen is performed by the ALPHA technology based on the principle described below using two types of beads: donor and acceptor beads. A luminescent signal is detected only when the two beads are located in close proximity through the biological interaction between the molecules linked to the donor beads and the acceptor beads. Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fc gamma receptor is immobilized to the acceptor beads. In the absence of an antigen-binding molecule or antibody comprising a competitive mutant Fc domain, Fc gamma receptor interacts with an antigen-binding molecule or antibody comprising a wild-type Fc domain, inducing a signal of 520 to 620 nm as a result. When an antigen-binding molecule or antibody having a non-tagged mutant Fc domain competes with the antigen-binding molecule or antibody comprising a wild-type Fc domain for the interaction with Fc gamma receptor, reduction of fluorescence will be observed as a result of competition and the reduction can be quantified to thereby determine the relative binding affinity. Methods for biotinylating the antigen-binding molecules or antibodies such as antibodies using Sulfo-NHS-biotin or the like are known. Appropriate methods for adding the GST tag to an Fc gamma receptor include methods that involve the steps of fusing genes encoding Fc gamma receptor and GST in-frame, expressing the fused gene using cells to which a vector carrying the fused gene is introduced, and then purifying the fused protein using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto a gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE methods. Examples of such inhibition assay are described in Proc. Natl. Acad. Sci. USA (2006) 103(11), 4005-4010.

Fc Region with a Reduced Fc Gamma Receptor-Binding Activity

Herein, “a reduced Fc gamma receptor-binding activity” means, for example, that based on the above-described analysis method the competitive activity of a test antigen-binding molecule or antibody is 50% or less, preferably 45% or less, 40% or less, 35% or less, 30% or less, 20% or less, or 15% or less, and particularly preferably 10% or less, 9% or less, 8% or less, 7% or less, 6% or less, 5% or less, 4% or less, 3% or less, 2% or less, or 1% or less than the competitive activity of a control antigen-binding molecule or antibody.

Antigen-binding molecules or antibodies comprising the Fc domain of a monoclonal IgG1, IgG2, IgG3, or IgG4 antibody can be appropriately used as control antigen-binding molecules or antibodies. The Fc domain structures are shown in SEQ ID NOs: 19 (A is added to the N terminus of RefSeq accession number AAC82527.1), 20 (A is added to the N terminus of RefSeq accession number AAB59393.1), 21 (RefSeq accession number CAA27268.1), and 22 (A is added to the N terminus of RefSeq accession number AAB59394.1). Furthermore, when an antigen-binding molecule or antibody comprising an Fc domain mutant of an antibody of a particular isotype is used as a test substance, the effect of the mutation of the mutant on the Fc gamma receptor-binding activity is assessed using as a control an antigen-binding molecule or antibody comprising an Fc domain of the same isotype. As described above, antigen-binding molecules or antibodies comprising an Fc domain mutant whose Fc gamma receptor-binding activity has been judged to be reduced are appropriately prepared.

Such known mutants include, for example, mutants having a deletion of amino acids 231A-238S (EU numbering) (WO 2009/011941), as well as mutants C226S, C229S, P238S, (C220S) (J. Rheumatol (2007) 34, 11); C226S and C229S (Hum. Antibod. Hybridomas (1990) 1(1), 47-54); C226S, C229S, E233P, L234V, and L235A (Blood (2007) 109, 1185-1192).

Specifically, the preferred antigen-binding molecules or antibodies include those comprising an Fc domain with a mutation (such as substitution) of at least one amino acid selected from the following amino acid positions: 220, 226, 229, 231, 232, 233, 234, 235, 236, 237, 238, 239, 240, 264, 265, 266, 267, 269, 270, 295, 296, 297, 298, 299, 300, 325, 327, 328, 329, 330, 331, or 332 (EU numbering), in the amino acids forming the Fc domain of an antibody of a particular isotype. The isotype of antibody from which the Fc domain originates is not particularly limited, and an appropriate Fc domain derived from a monoclonal IgG1, IgG2, IgG3, or IgG4 antibody may be used. It is preferable to use Fc domains derived from IgG1 antibodies.

The preferred antigen-binding molecules or antibodies include, for example, those comprising an Fc domain which has any one of the substitutions shown below, whose positions are specified according to EU numbering (each number represents the position of an amino acid residue in the EU numbering; and the one-letter amino acid symbol before the number represents the amino acid residue before substitution, while the one-letter amino acid symbol after the number represents the amino acid residue after the substitution) in the amino acids forming the Fc domain of IgG1 antibody:
(a) L234F, L235E, P331S;
(b) C226S, C229S, P238S;
(c) C226S, C229S; or
(d) C226S, C229S, E233P, L234V, L235A;
   as well as those having an Fc domain which has a deletion of the amino acid sequence at positions 231 to 238.

Furthermore, the preferred antigen-binding molecules or antibodies also include those comprising an Fc domain that has any one of the substitutions shown below, whose positions are specified according to EU numbering in the amino acids forming the Fc domain of an IgG2 antibody:
(e) H268Q, V309L, A330S, and P331S;
(f) V234A;
(g) G237A;
(h) V234A and G237A;
(i) A235E and G237A; or
(j) V234A, A235E, and G237A. Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG3 antibody:
(k) F241A;
(l) D265A; or
(m) V264A. Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG4 antibody:
(n) L235A, G237A, and E318A;
(o) L235E; or
(p) F234A and L235A. Each number represents the position of an amino acid residue in EU numbering; and the one-letter amino acid symbol before the number represents the amino acid residue before substitution, while the one-letter amino acid symbol after the number represents the amino acid residue after the substitution.

The other preferred antigen-binding molecules or antibodies include, for example, those comprising an Fc domain in which any amino acid at position 233, 234, 235, 236, 237, 327, 330, or 331 (EU numbering) in the amino acids forming the Fc domain of an IgG1 antibody is substituted with an amino acid of the corresponding position in EU numbering in the corresponding IgG2 or IgG4.

The preferred antigen-binding molecules or antibodies also include, for example, those comprising an Fc domain in which any one or more of the amino acids at positions 234, 235, and 297 (EU numbering) in the amino acids forming the Fc domain of an IgG1 antibody is substituted with other amino acids. The type of amino acid after substitution is not particularly limited; however, the antigen-binding molecules or antibodies comprising an Fc domain in which any one or more of the amino acids at positions 234, 235, and 297 are substituted with alanine are particularly preferred.

The preferred antigen-binding molecules or antibodies also include, for example, those comprising an Fc domain in which an amino acid at position 265 (EU numbering) in the amino acids forming the Fc domain of an IgG1 antibody is substituted with another amino acid. The type of amino acid after substitution is not particularly limited; however, antigen-binding molecules or antibodies comprising an Fc domain in which an amino acid at position 265 is substituted with alanine are particularly preferred.

### Antigen-Binding Domains Binding to PDGF-B

The phrase “an antigen-binding domain (that) binds to PDGF-B” or “an anti-PDGF-B antigen-binding domain” as used herein refers to an antigen-binding domain that specifically binds to the above-mentioned PDGF-B protein, or the whole or a portion of a partial peptide of the PDGF-B protein.

In certain embodiments, the antigen-binding domain that binds to PDGF-B comprises an antibody variable region (antibody light-chain and heavy-chain variable regions (VL and VH)). Suitable examples of domains comprising antibody light-chain and heavy-chain variable regions include “single chain Fv (scFv)”, “single chain antibody”, “Fv”, “single chain Fv₂ (scFv₂)”, “Fab”, “F(ab′)2”, etc. In specific embodiments, the antigen-binding domain that binds to PDGF-B comprises an antibody variable fragment. Domains comprising an antibody variable fragment may be provided from variable domains of one or a plurality of antibodies.

In certain embodiments, the antigen-binding domain that binds to PDGF-B comprises the heavy-chain variable region and light-chain variable region of an anti-PDGF-B antibody. In certain embodiments, the antigen-binding domain that binds to PDGF-B comprises a Fab structure.

Preferably, the anti-PDGF-B antibody comprises an H chain comprising the amino acid sequence (of the H-chain variable region) as described in Table A and an L chain comprising the amino acid sequence (of the L-chain variable region) as described in the Table A.

In specific embodiments, the antigen-binding domain that binds to PDGF-B comprises any one of the antibody variable fragments shown in Table A below.

TABLE A
Sequences of HVRs (CDRs) or VH, VL in an antigen-binding domain that binds to PDGF-B

| | SEQ ID NO: | | | | | | | |
|---|---|---|---|---|---|---|---|---|
| Antibody | Heavy chain | HVR- | HVR- | HVR- | Light chain | HVR- | HVR- | HVR- |
| Name | variable region | H1 | H2 | H3 | variable region | L1 | L2 | L3 |
| CPR001 | 1 | 5 | 6 | 7 | 2 | 8 | 9 | 10 |

In specific embodiments, the antigen-binding domain that binds to PDGF-B comprises an antibody variable fragment that competes for binding to human PDGF-B with any one of the antibody variable fragments shown in Table A.

Alternatively, the antigen-binding domain that binds to PDGF-B comprises an antibody variable fragment that competes for binding to human PDGF-B with any one of the above-mentioned antibody variable fragments. Alternatively, the antigen-binding domain that binds to PDGF-B comprises an antibody variable fragment that binds to the same epitope on human PDGF-B to which any one of the above-mentioned antibody variable fragments binds.

### Antigen-Binding Domains Binding to PDGF-D

The phrase “an antigen-binding domain (that) binds to PDGF-D” or “an anti-PDGF-D antigen-binding domain” as used herein refers to an antigen-binding domain that specifically binds to the above-mentioned PDGF-D protein, or the whole or a portion of a partial peptide of the PDGF-D protein.

In certain embodiments, the antigen-binding domain that binds to PDGF-D comprises an antibody variable region (antibody light-chain and heavy-chain variable regions (VL and VH)). Suitable examples of domains comprising antibody light-chain and heavy-chain variable regions include “single chain Fv (scFv)”, “single chain antibody”, “Fv”, “single chain Fv₂ (scFv₂)”, “Fab”, “F(ab′)₂”, etc. In specific embodiments, the antigen-binding domain that binds to PDGF-D comprises an antibody variable fragment. Domains comprising an antibody variable fragment may be provided from variable domains of one or a plurality of antibodies.

In certain embodiments, the antigen-binding domain that binds to PDGF-D comprises the heavy-chain variable region and light-chain variable region of an anti-PDGF-D antibody. In certain embodiments, the antigen-binding domain that binds to PDGF-D comprises a Fab structure.

Preferably, the anti-PDGF-D antibody comprises an H chain comprising the amino acid sequence (of the H-chain variable region) as described in Table B and an L chain comprising the amino acid sequence (of the L-chain variable region) as described in Table B.

In specific embodiments, the antigen-binding domain that binds to PDGF-D comprises any one of the antibody variable fragments shown in Table B below.

TABLE B
Sequences of HVRs (CDRs) or VH, VL in an antigen-binding domain that binds to PDGF-D

| | SEQ ID NO: | | | | | | | |
|---|---|---|---|---|---|---|---|---|
| Antibody | Heavy chain | HVR- | HVR- | HVR- | Light chain | HVR- | HVR- | HVR- |
| Name | variable region | H1 | H2 | H3 | variable region | L1 | L2 | L3 |
| CPR002 | 3 | 11 | 12 | 13 | 4 | 14 | 15 | 16 |

In specific embodiments, the antigen-binding domain that binds to PDGF-D comprises an antibody variable fragment that binds to the same epitope within human PDGF-D as any one of the antibody variable fragments shown in Table B.

Alternatively, the antigen-binding domain that binds to PDGF-D comprises an antibody variable fragment that competes for binding to human PDGF-D with any one of the above-mentioned antibody variable fragments. Alternatively, the antigen-binding domain that binds to PDGF-D comprises an antibody variable fragment that binds to the same epitope on human PDGF-D to which any one of the above-mentioned antibody variable fragments binds.

In another aspect, the present invention further relates to an antigen-binding molecule that specifically binds to PDGF-D and blocks its interaction with Neuropilin 1 (NRP1). NRP1 binds to PDGF-D and is a co-receptor in PDGF-D-PDGFR-beta signalling (Muhl, Lars, et al., J Cell Sci 130.8 (2017): 1365-1378). In one embodiment, the antigen-binding molecule is an antibody that specifically binds to PDGF-D and blocks/inhibits its interaction with Neuropilin 1 (NRP1) and also blocks/inhibits PDGF-D binding to PDGFR, thereby inhibits the PDGF-D-induced signalling. Such antibody is expected to show enhanced inhibition of PDGF-D-mediated signalling compared to anti-PDGF-D antibody that is not capable of blocking NRP1-PDGF-D interaction, for use as a more effective anti-PDGF-D antibody for treating/preventing a PDGF-D-mediated disease/condition. Method of obtaining an antibody that specifically binds to PDGF-D and blocks/inhibits its interaction with NRP1 and also blocks/inhibits PDGF-D binding to PDGFR are obtained via known antibody immunization followed by evaluation and screening for inhibition of NRP1-PDGF-D interaction using well-known method such as ELISA, Octet, Biacore and/or ECL and so on.

### Multispecific Antigen-Binding Molecules

“Multispecific antigen-binding molecules” refers to antigen-binding molecules that bind specifically to more than one antigen. In a favorable embodiment, multispecific antigen-binding molecules of the present invention comprise two or more antigen-binding domains, and different antigen-binding domains bind specifically to different antigens.

The multispecific antigen-binding molecule of the present invention comprises a first antigen-binding domain that binds to PDGF-B, and a second antigen-binding domain that binds to PDGF-B. The combinations of an antigen-binding domain that binds to PDGF-B selected from those described in “Antigen-binding domains binding to PDGF-B” above and an antigen-binding domain that binds to PDGF-D selected from those described in “Antigen-binding domains binding to PDGF-D” above can be used.

For example, the first antigen-binding domain comprises antibody heavy-chain and light-chain variable regions, and/or the second antigen-binding domain comprises antibody heavy-chain and light-chain variable regions. Alternatively, the first antigen-binding domain comprises an antibody variable fragment, and/or the second antigen-binding domain comprises an antibody variable fragment. Alternatively, the first antigen-binding domain comprises a Fab structure, and/or the second antigen-binding domain comprises a Fab structure.

In certain embodiments, the present invention provides multispecific antigen-binding molecules comprising a first antigen-binding domain that comprises an antibody variable fragment that binds to PDGF-B, and a second antigen-binding domain that comprises an antibody variable fragment that binds to PDGF-D. In certain embodiments, the present invention provides bispecific antigen-binding molecules that comprise a first antigen-binding domain that binds to PDGF-B, a second antigen-binding domain that binds to PDGF-D, and a third domain comprising an Fc region that has a reduced Fc gamma receptor-binding activity. The Fc region may have a reduced Fc gamma receptor-binding activity compared with the Fc domain of an IgG1, IgG2, IgG3, or IgG4 antibody.

In certain embodiments, the present invention provides bispecific antibodies that comprise a first antibody variable fragment that binds to human PDGF-B, and a second antibody variable fragment that binds to human PDGF-D. In certain embodiments, the present invention provides bispecific antibodies that comprise a first antibody variable fragment that binds to human PDGF-B, a second antibody variable fragment that binds to human PDGF-D, and an Fc region that has a reduced Fc gamma receptor-binding activity. In certain embodiments, the present invention provides bispecific antibodies that comprise a first antibody variable fragment that binds to human PDGF-B, a second antibody variable fragment that binds to human PDGF-D, and an Fc region that has a reduced Fc gamma receptor-binding activity compared with naturally occurring IgG Fc regions.

Examples of a preferred embodiment of the “multispecific antigen-binding molecule” of the present invention include multispecific antibodies. When an Fc region with reduced Fc gamma receptor-binding activity is used as the multispecific antibody Fc region, an Fc region derived from the multispecific antibody may be used appropriately. Bispecific antibodies are particularly preferred as the multispecific antibodies of the present invention. In this case, a bispecific antibody is an antibody having two different specificities. IgG-type bispecific antibodies can be secreted from a hybrid hybridoma (quadroma) produced by fusing two types of hybridomas that produce IgG antibodies (Milstein et al., Nature (1983) 305, 537-540).

Furthermore, IgG-type bispecific antibodies are secreted by introducing the genes of L chains and H chains constituting the two types of IgGs of interest, i.e., a total of four genes, into cells, and co-expressing them. However, the number of combinations of H and L chains of IgG that can be produced by these methods is theoretically ten combinations. Accordingly, it is difficult to purify an IgG comprising the desired combination of H and L chains from ten types of IgGs. Furthermore, theoretically, the amount of secretion of the IgG having the desired combination will decrease remarkably, and therefore large-scale culturing will be necessary, and production costs will increase further.

Therefore, techniques for promoting the association among H chains and between L and H chains having the desired combinations can be applied to the multispecific antigen-binding molecules of the present invention.

For example, techniques for suppressing undesired H-chain association by introducing electrostatic repulsion at the interface of the second constant region or the third constant region of the antibody H chain (CH2 or CH3) can be applied to multispecific antibody association (WO2006/106905).

In the technique of suppressing unintended H-chain association by introducing electrostatic repulsion at the interface of CH2 or CH3, examples of amino acid residues in contact at the interface of the other constant region of the H chain include regions corresponding to the residues at EU numbering positions 356, 439, 357, 370, 399, and 409 in the CH3 region.

More specifically, examples include an antibody comprising two types of H-chain CH3 regions, in which one to three pairs of amino acid residues in the first H-chain CH3 region, selected from the pairs of amino acid residues indicated in (1) to (3) below, carry the same type of charge: (1) amino acid residues comprised in the H chain CH3 region at EU numbering positions 356 and 439; (2) amino acid residues comprised in the H-chain CH3 region at EU numbering positions 357 and 370; and (3) amino acid residues comprised in the H-chain CH3 region at EU numbering positions 399 and 409.

Furthermore, the antibody may be an antibody in which pairs of the amino acid residues in the second H-chain CH3 region which is different from the first H-chain CH3 region mentioned above, are selected from the aforementioned pairs of amino acid residues of (1) to (3), wherein the one to three pairs of amino acid residues that correspond to the aforementioned pairs of amino acid residues of (1) to (3) carrying the same type of charges in the first H-chain CH3 region mentioned above carry opposite charges from the corresponding amino acid residues in the first H-chain CH3 region mentioned above.

Each of the amino acid residues indicated in (1) to (3) above come close to each other during association. Those skilled in the art can find out positions that correspond to the above-mentioned amino acid residues of (1) to (3) in a desired H-chain CH3 region or H-chain constant region by homology modeling and such using commercially available software, and amino acid residues of these positions can be appropriately subjected to modification.

In the antibodies mentioned above, “charged amino acid residues” are preferably selected, for example, from amino acid residues included in either one of the following groups:
(a) glutamic acid (E) and aspartic acid (D); and
(b) lysine (K), arginine (R), and histidine (H).

In the above-mentioned antibodies, the phrase “carrying the same charge” means, for example, that all of the two or more amino acid residues are selected from the amino acid residues included in either one of groups (a) and (b) mentioned above. The phrase “carrying opposite charges” means, for example, that when at least one of the amino acid residues among two or more amino acid residues is selected from the amino acid residues included in either one of groups (a) and (b) mentioned above, the remaining amino acid residues are selected from the amino acid residues included in the other group.

In a preferred embodiment, the antibodies mentioned above may have their first H-chain CH3 region and second H-chain CH3 region crosslinked by disulfide bonds.

In the present invention, amino acid residues subjected to modification are not limited to the above-mentioned amino acid residues of the antibody variable regions or the antibody constant regions. Those skilled in the art can identify the amino acid residues that form an interface in mutant polypeptides or heteromultimers by homology modeling and such using commercially available software; and amino acid residues of these positions can then be subjected to modification so as to regulate the association.

Other known techniques can also be used for the association of multispecific antibodies of the present invention. Fc region-containing polypeptides comprising different amino acids can be efficiently associated with each other by substituting an amino acid side chain present in one of the H-chain Fc regions of the antibody with a larger side chain (knob), and substituting an amino acid side chain present in the corresponding Fc region of the other H chain with a smaller side chain (hole) to allow placement of the knob within the hole (WO1996/027011; Ridgway J B et al., Protein Engineering (1996) 9, 617-621; Merchant A. M. et al. Nature Biotechnology (1998) 16, 677-681; and US20130336973).

In addition, other known techniques can also be used for formation of multispecific antibodies of the present invention. Association of polypeptides having different sequences can be induced efficiently by complementary association of CH3 using a strand-exchange engineered domain CH3 produced by changing part of one of the H-chain CH3s of an antibody to a corresponding IgA-derived sequence and introducing a corresponding IgA-derived sequence into the complementary portion of the other H-chain CH3 (Protein Engineering Design & Selection, 23; 195-202, 2010). This known technique can also be used to efficiently form multispecific antibodies of interest.

In addition, technologies for antibody production using association of antibody CH1 and CL and association of VH and VL as described in WO 2011/028952, WO2014/018572, and Nat Biotechnol. 2014 February; 32(2):191-8; technologies for producing bispecific antibodies using separately prepared monoclonal antibodies in combination (Fab Arm Exchange) as described in WO2008/119353, WO2011/131746, WO2015/046467 and WO2016159213; technologies for regulating association between antibody heavy-chain CH3s as described in WO2012/058768 and WO2013/063702; technologies for producing bispecific antibodies composed of two types of light chains and one type of heavy chain as described in WO2012/023053; technologies for producing bispecific antibodies using two bacterial cell strains that individually express one of the chains of an antibody comprising a single H chain and a single L chain as described by Christoph et al. (Nature Biotechnology Vol. 31, p 753-758 (2013)); and such may be used for the formation of multispecific antibodies.

Alternatively, even when a multispecific antibody of interest cannot be formed efficiently, a multispecific antibody of the present invention can be obtained by separating and purifying the multispecific antibody of interest from the produced antibodies. For example, a method for enabling purification of two types of homomeric forms and the heteromeric antibody of interest by ion-exchange chromatography by imparting a difference in isoelectric points by introducing amino acid substitutions into the variable regions of the two types of H chains has been reported (WO2007114325). To date, as a method for purifying heteromeric antibodies, methods using Protein A to purify a heterodimeric antibody comprising a mouse IgG2a H chain that binds to Protein A and a rat IgG2b H chain that does not bind to Protein A have been reported (WO98050431 and WO95033844). Furthermore, a heterodimeric antibody can be purified efficiently on its own by using H chains comprising substitution of amino acid residues at EU numbering positions 435 and 436, which is the IgG-Protein A binding site, with Tyr, His, or such which are amino acids that yield a different Protein A affinity, or using H chains with a different protein A affinity, to change the interaction of each of the H chains with Protein A, and then using a Protein A column.

Furthermore, an Fc region whose Fc region C-terminal heterogeneity has been improved can be appropriately used as an Fc region of the present invention. More specifically, the present invention provides Fc regions produced by deleting glycine at position 446 and lysine at position 447 as specified by EU numbering from the amino acid sequences of two polypeptides constituting an Fc region derived from IgG1, IgG2, IgG3, or IgG4.

A plurality, such as two or more, of these technologies can be used in combination. Furthermore, these technologies can be appropriately and separately applied to the two H chains to be associated. Furthermore, these techniques can be used in combination with the above-mentioned Fc region which has reduced binding activity to an Fc gamma receptor. Furthermore, an antigen-binding molecule of the present invention may be a molecule produced separately so that it has the same amino acid sequence, based on the antigen-binding molecule subjected to the above-described modifications.

Preferably, the antigen-binding molecule of the present invention is a multispecific antigen-binding molecule comprising a first antigen-binding domain that binds to PDGF-B, and a second antigen-binding domain that binds to PDGF-D. More preferably, the antigen-binding molecule of the present invention specifically binds to PDGF-B (i.e., PDGF-B, PDGF-AB and PDGF-BB) and PDGF-D (i.e., PDGF-D and PDGF-DD), and inhibits their interaction with PDGFR, thereby inhibiting PDGF-B activity and PDGF-D activity.

By the terms “PDGF-B mediated activity”, “PDGF-B mediated effect”, “PDGF-B activity”, “PDGF-B biological activity”, or “PDGF-B function”, as used interchangeably herein, is meant any activity mediated by PDGF-B interaction with a cognate receptor including, but not limited to, binding of PDGF-B to PDGFR, phosphorylation of PDGFR, increase in cell migration, increase in cell proliferation, increase in extracellular matrix deposition, and any other activity of PDGF-B either known in the art or to be elucidated in the future. In one embodiment, the antigen-binding molecule of the present invention is an antibody that specifically binds to PDGF-B. In one embodiment, the extent of binding of the antigen-binding molecule/antibody of the present invention to an unrelated, non-PDGF-B protein is less than about 10% of the binding of the antibody to PDGF-B as measured, e.g., by a radioimmunoassay (RIA). In one embodiment, said non-PDGF-B is PDGF-A, PDGF-C, or PDGF-D. In certain embodiments, an antibody that binds to PDGF-B has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M). In certain embodiments, an anti-PDGF-B antibody binds to an epitope of PDGF-B that is conserved among PDGF-B from different species.

By the terms “PDGF-D mediated activity”, “PDGF-D mediated effect”, “PDGF-D activity”, “PDGF-D biological activity”, or “PDGF-Dfunction”, as used interchangeably herein, is meant any activity mediated by PDGF-D interaction with a cognate receptor including, but not limited to, binding of PDGF-D to PDGFR, phosphorylation of PDGFR, increase in cell migration, increase in cell proliferation, increase in extracellular matrix deposition, and any other activity of PDGF-D either known in the art or to be elucidated in the future. In one embodiment, the antigen-binding molecule of the present invention is an antibody that specifically binds to PDGF-D. In one embodiment, the extent of binding of the antigen-binding molecule/antibody of the present invention to an unrelated, non-PDGF-D protein is less than about 10% of the binding of the antibody to PDGF-D as measured, e.g., by a radioimmunoassay (RIA). In one embodiment, said non-PDGF-D is PDGF-A, PDGF-B, or PDGF-C. In certain embodiments, an antibody that binds to PDGF-D has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M). In certain embodiments, an anti-PDGF-D antibody binds to an epitope of PDGF-D that is conserved among PDGF-D from different species.

Thus, the methods of the present invention use the multipecific antigen-binding molecule or antibody of the present invention that blocks, suppresses, or reduces (including significantly reduces) PDGF-B and/or PDGF-D activity, including downstream events mediated by PDGF-B and/or PDGF-D. A multipecific antigen-binding molecule or antibody of the present invention exhibits any one or more of the following characteristics: (a) specifically binding to PDGF-B and/or PDGF-D; (b) blocking PDGF-B and/or PDGF-D interaction with a cell surface receptor and downstream signaling events; (c) blocking phosphorylation of the PDGFR; (d) blocking PDGF-B and/or PDGF-D mediated induction of cell proliferation; (e) blocking PDGF-B and/or PDGF-D mediated induction of cell migration; and (f) blocking or reducing PDGF-B and/or PDGF-D mediated deposition of extracellular matrix. In one preferred embodiment, the antigen-binding molecule or antibody of the present invention preferably reacts with PDGF-B and/or PDGF-D in a manner where PDGF-B and/or PDGF-D interaction with a cell surface receptor, e.g., PDGFR, is blocked.

In one preferred embodiment, the multispecific antigen-binding molecule of the present invention comprises one or more polypeptide chains as listed in Table 1 and Table 2.

### Pharmaceutical Formulations

Pharmaceutical formulations of an antigen-binding molecule (e.g. antibody) binding to PDGF-B and/or PDGF-D as described herein are prepared by mixing such an antigen-binding molecule (e.g. antibody) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### Therapeutic Methods and Compositions

Any of the antigen-binding molecule (e.g. antibody) binding to PDGF-B and/or PDGF-D provided herein may be used in therapeutic methods. In one aspect, an antigen-binding molecule (e.g. antibody) binding to PDGF-B, an antigen-binding molecule (e.g. antibody) binding to PDGF-D, alone or in combination, for use as a medicament is provided. In another aspect, a multispecific antigen-binding molecule comprising a first antigen-binding domain that binds to PDGF-B and a second antigen-binding domain that binds to PDGF-D, for use as a medicament is provided.

In one aspect, an antigen-binding molecule (e.g. antibody) binding to PDGF-B, an antigen-binding molecule (e.g. antibody) binding to PDGF-D, alone or in combination, for use in treating fibrosis (such as myocardial fibrosis, pulmonary fibrosis, liver fibrosis, renal fibrosis, skin fibrosis, ocular fibrosis and myelofibrosis), or nephritis and related diseases in humans, including but not limited to, nephritis, progressive renal diseases, and related diseases, such as IgA nephropathy, mesangial proliferative nephritis, mesangial proliferative glomerulonephritis, mesangiocapillary glomerulonephritis, systemic lupus erythematosus, glomerular nephritis, renal interstitial fibrosis, renal failure, diabetic nephropathy, polycystic kidney disease, alport syndrome, focal segmental glomerular sclerosis, membranous nephropathy is provided. In another aspect, a multispecific antigen-binding molecule comprising a first antigen-binding domain that binds to PDGF-B and a second antigen-binding domain that binds to PDGF-D, for use in treating the abovementioned diseases/conditions is provided.

In certain embodiments, the invention provides an antigen-binding molecule (e.g. antibody) binding to PDGF-B, an antigen-binding molecule (e.g. antibody) binding to PDGF-D, alone or in combination, for use in a method of treating an individual having fibrosis (such as myocardial fibrosis, pulmonary fibrosis, liver fibrosis, renal fibrosis, skin fibrosis, ocular fibrosis and myelofibrosis), or nephritis and related diseases in humans, including but not limited to, nephritis, progressive renal diseases, and related diseases, such as IgA nephropathy, mesangial proliferative nephritis, mesangial proliferative glomerulonephritis, mesangiocapillary glomerulonephritis, systemic lupus erythematosus, glomerular nephritis, renal interstitial fibrosis, renal failure, diabetic nephropathy, polycystic kidney disease, alport syndrome, focal segmental glomerular sclerosis, membranous nephropathy; wherein said method comprises administering to the individual an effective amount of the antigen-binding molecule of the present invention. An “individual” according to any of the above embodiments is preferably a human. In another aspect, the invention provides a multispecific antigen-binding molecule comprising a first antigen-binding domain that binds to PDGF-B and a second antigen-binding domain that binds to PDGF-D, for use in any of the above therapeutic methods.

In a further aspect, the invention provides use of an antigen-binding molecule (e.g. antibody) binding to PDGF-B and/or PDGF-D in the manufacture or preparation of a medicament. In one embodiment, the medicament is for treating fibrosis (such as myocardial fibrosis, pulmonary fibrosis, liver fibrosis, renal fibrosis, skin fibrosis, ocular fibrosis and myelofibrosis), or nephritis and related diseases in humans, including but not limited to, nephritis, progressive renal diseases, and related diseases, such as IgA nephropathy, mesangial proliferative nephritis, mesangial proliferative glomerulonephritis, mesangiocapillary glomerulonephritis, systemic lupus erythematosus, glomerular nephritis, renal interstitial fibrosis, renal failure, diabetic nephropathy, polycystic kidney disease, alport syndrome, focal segmental glomerular sclerosis, membranous nephropathy. In another aspect, the invention provides a multispecific antigen-binding molecule comprising a first antigen-binding domain that binds to PDGF-B and a second antigen-binding domain that binds to PDGF-D, for use in the manufacture or preparation of a medicament for treating any of the above diseases/conditions.

In a further aspect, the invention provides a pharmaceutical formulation comprising an antigen-binding molecule which binds to PDGF-B and an antigen-binding molecule which binds to PDGF-D provided herein, e.g., for use in any of the above therapeutic methods. In one embodiment, the invention provides a a pharmaceutical composition comprising an antigen-binding molecule which binds to PDGF-B in combination with a pharmaceutical composition comprising an antigen-binding molecule which binds to PDGF-D. In some embodiments, for the pharmaceutical composition provided herein, the antigen-binding molecule which binds to PDGF-B is administered to the subject simultaneously, separately, or sequentially with the antigen-binding molecule which binds to PDGF-D. In a further aspect, the invention provides a pharmaceutical formulation comprising a multispecific antigen-binding molecule comprising a first antigen-binding domain that binds to PDGF-B and a second antigen-binding domain that binds to PDGF-D, for use in any of the above therapeutic methods.

In another embodiment, a pharmaceutical formulation comprises any of the antigen-binding molecule (e.g. antibody) binding to PDGF-B and/or PDGF-D provided herein and at least one additional therapeutic agent. Such combination therapies noted above encompass combined administration (where two or more therapeutic agents are included in the same or separate formulations), and separate administration, in which case, administration of the antibody of the present invention can occur prior to, simultaneously, and/or following, administration of the additional therapeutic agent or agents. In one embodiment, administration of the antigen-binding molecule (e.g. antibody) binding to PDGF-B and/or PDGF-D provided herein and administration of an additional therapeutic agent occur within about one month, or within about one, two or three weeks, or within about one, two, three, four, five, or six days, of each other.

An antibody of the present invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the present invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody needs not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the present invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 micro g/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

### Articles of Manufacture

In another aspect of the present invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antigen-binding molecule or an antibody of the present invention. The label or package insert indicates that the composition is used for treating the condition of choice. Moreover, the article of manufacture may comprise (a) a first container with a composition contained therein, wherein the composition comprises an antigen-binding molecule or an antibody of the present invention; and (b) a second container with a composition contained therein, wherein the composition comprises a further cytotoxic or otherwise therapeutic agent. The article of manufacture in this embodiment of the present invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of methods and compositions of the present invention. It should be understood that various other embodiments may be practiced, in view of the general description provided above.

### Example 1

### Antibody Preparation

The monospecific antibody against PDGF-B (CPR001; Tables 1 and 2), monospecific antibody against PDGF-D (CR002; Tables 1 and 2), and bispecific antibody against PDGF-B and PDGF-D (comprising anti-PDGF-B arm of CPR001 and anti-PDGF-D arm of CR002) were expressed using an Expi293 Expression system (Thermo Fisher Scientific) by transient transfection of genes coding therefor. Culture supernatants were harvested, and antibodies were purified from the supernatants using MabSelect SuRe affinity chromatography (GE Healthcare) followed by gel filtration chromatography using Superdex200 (GE Healthcare). Bispecific antibody against PDGF-B and PDGF-D (i.e. CPR//CR) was prepared using the anti-PDGF-B arm of CPR001 and anti-PDGF-D arm of CR002 via conventional methods such as published in WO2015046467A1 or Sci Rep. 2017 Apr. 3; 7:45839.

TABLE 1
Names and SED ID NOs of variable regions (including VH, VL, and HVR/CDR) of monospecific
antibody against PDGF-B (CPR001) and monospecific antibody against PDGF-D (CR002).

| | SEQ ID NO: | | | | | | | |
|---|---|---|---|---|---|---|---|---|
| | Heavy chain | | | | Light chain | | | |
| Antibody | variable region | HVR- | HVR- | HVR- | variable region | HVR- | HVR- | HVR- |
| Name | (VH) | H1 | H2 | H3 | (VL) | L1 | L2 | L3 |
| CPR001 | 1 | 5 | 6 | 7 | 2 | 8 | 9 | 10 |
| CR002 | 3 | 11 | 12 | 13 | 4 | 14 | 15 | 16 |

TABLE 2
Names and amino acid sequences of variable regions (including
VH, VL, and HVR/CDR) of monospecific antibody against PDGF-B (CPRO01)
and monospecific antibody against PDGF-D (CR002).

| Antibody | | | |
|---|---|---|---|
| Name | VR | Amino acid sequence | SEQ ID NO |
| CPR001 | VH | EVQLLESGGGLVQPGGSLRLSCAASGFT | 1 |
| | | FSSYAMSWVRQAPGKGLEWVSYISDDG | |
| | | SLKYYADSVKGRFTISRDNSKNTLYLQM | |
| | | NSLRAEDTAVYYCAKHPYWYGGQLDL | |
| | | WGQGTLVTVSS | |
| CPR001 | VL | SYELTQPPSVSVSPGQTASITCSGDSLGS | 2 |
| | | YFVHWYQQKPGQSPVLVIYDDSNRPSGI | |
| | | PERFSGSNSGNTATLTISGTQAMDEADY | |
| | | YCSAFTHNSDVFGGGTKLTVL | |
| CR002 | VH | QVQLVQSGAEVKKPGASVKVSCKASGY | 3 |
| | | TFTSYDINWVRQATGQGLEWMGWINPN | |
| | | SGNTDYAQKFQGRVTMTRDTSISTAYM | |
| | | ELSSLRSEDTAIYYCVRGFGYSYNYDYY | |
| | | YGMDVWGQGTTVTVSS | |
| CR002 | VL | EIVLTQSPGTLSLSPGERATLSCRASQSV | 4 |
| | | SSSYLAWYQQKPGQAPRLLIYATSSRAT | |
| | | GIPDRFSGSGSGTDFTLTISRLEPEDFAVY | |
| | | YCOQYGSSPCSFGQGTKLEIK | |
| CPR001 | HVR H1 | SYAMS | 5 |
| CPR001 | HVR H2 | YISDDGSLKYYADSVKG | 6 |
| CPR001 | HVR H3 | HPYWYGGQLDL | 7 |
| CPR001 | HVR L1 | SGDSLGSYFVH | 8 |
| CPR001 | HVR L2 | DDSNRPS | 9 |
| CPR001 | HVR L3 | SAFTHNSDV | 10 |
| CR002 | HVR H1 | SYDIN | 11 |
| CR002 | HVR H2 | WINPNSGNTDYAQKFQG | 12 |
| CR002 | HVR H3 | GFGYSYNYDYYYGMDV | 13 |
| CR002 | HVR L1 | RASQSVSSSYLA | 14 |
| CR002 | HVR L2 | ATSSRAT | 15 |
| CR002 | HVR L3 | QQYGSSPCS | 16 |

### Example 2

### Characterization of Anti-PDGF Antibodies

### 2.1 Anti-PDGF Antibodies Inhibited PDGF-B and -D-Induced PDGFR Beta Phosphorylation in Mouse Fibroblast

Neutralization of recombinant mouse PDGF-B and -D by anti-PDGF antibodies (CPR001, CR002, or CPR//CR bi-specific antibody) was tested in mouse fibroblast cell line NIH3T3 by measuring PDGFR beta phosphorylation.

NIH3T3 cells were cultured and seeded onto 12-well plates and serum-starved under 0.1% FBS condition overnight. Cells were treated with antibodies and ligands (recombinant mouse PDGF-BB (Thermo) and/or recombinant mouse PDGF-D (R&D Systems) for 5 minutes at 37 degrees C. Different concentrations of antibodies were pre-incubated with 10 ng/mL of each ligands for 30 minutes at room temperature before treatment to cells. Phosphorylation of PDGFR beta was measured using PathScan (registered trademark) Phospho-PDGF Receptor beta (Tyr751) ELISA kit (Cell Signaling Technology) according to the manufacturer's procedure.

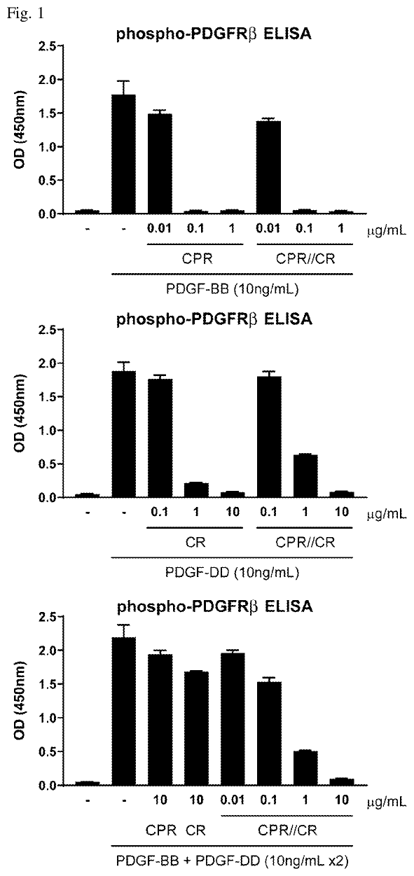

As shown in FIG. 1, CPR (i.e. CPR001) and CPR//CR inhibited PDGF-B-induced PDGFR beta phosphorylation, and CR (i.e. CR002) and CPR//CR inhibited PDGF-D-induced PDGFR beta phosphorylation in NIH3T3 cells. On the other hand, only CPR//CR inhibited PDGFR beta phosphorylation in the presence of PDGF-B and -D.

### 2.2 Anti-PDGF Antibodies Inhibited PDGF-B and -D-Induced PDGFR Alpha and PDGFR Beta Phosphorylation in Human Fibroblast

Neutralization of recombinant human PDGF-B and -D by anti-PDGF antibodies (CPR001, CR002, or CPR//CR bi-specific antibody) was tested in human lung fibroblast cell line IMR90 by measuring PDGFR alpha and beta phosphorylation.

IMR90 cells were cultured and seeded onto 12-well plates and serum-starved under 0.1% FBS condition overnight. Cells were treated with antibodies and ligands (recombinant human PDGF-BB and/or recombinant human PDGF-DD (R&D Systems)) for 5 minutes at 37 degrees C. Different concentrations of antibodies were pre-incubated with 10 ng/mL of each ligands for 30 minutes at room temperature before treatment to cells. Phosphorylation of PDGFR alpha and beta was measured using PathScan (registered trademark) Phospho-PDGF Receptor alpha (Tyr849) or Phospho-PDGF Receptor beta (Tyr751) ELISA kit (Cell Signaling Technology) according to manufacturer's procedure.

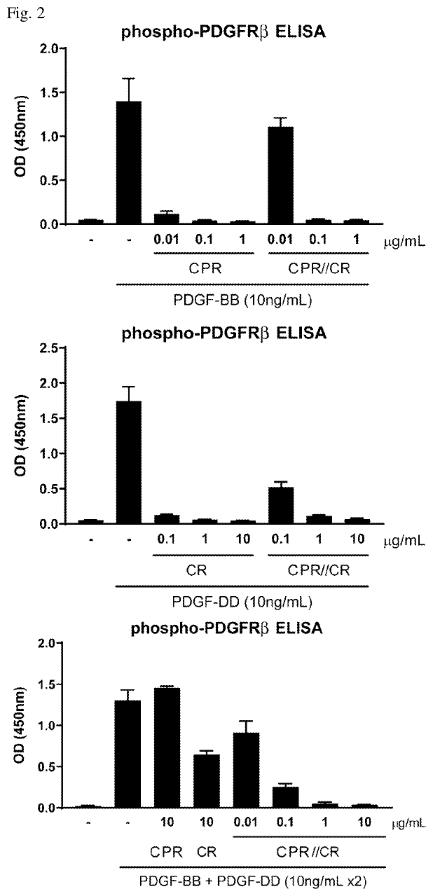

As shown in FIG. 2, CPR (i.e. CPR001) and CPR//CR inhibited PDGF-B-induced PDGFR beta phosphorylation, and CR (i.e. CR002) and CPR//CR inhibited PDGF-D-induced PDGFR beta phosphorylation in IMR90 cells. On the other hand, only CPR//CR inhibited PDGFR beta phosphorylation in the presence of PDGF-B and -D.

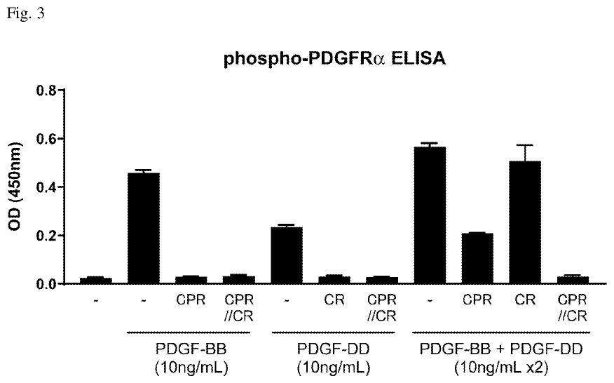

As shown in FIG. 3, CPR (i.e. CPR001) and CPR//CR inhibited PDGF-B-induced PDGFR alpha phosphorylation, and CR (i.e. CR002) and CPR//CR inhibited PDGF-D-induced PDGFR alpha phosphorylation in IMR90 cells. On the other hand, only CPR//CR inhibited PDGFR alpha phosphorylation in the presence of PDGF-B and -D.

### 2.3 Anti-PDGF Antibodies Inhibited PDGF-B and -D-Induced Proliferation of Mouse Fibroblast

Neutralization of recombinant mouse PDGF-B and -D by anti-PDGF antibodies (CPR001, CR002, or CPR//CR bi-specific antibody) was tested in mouse fibroblast cell line NIH3T3 by BrdU incorporation.

NIH3T3 cells were cultured and seeded onto 96-well plates and serum-starved under 0.1% FBS condition overnight. Cells were treated with antibodies and ligands (recombinant mouse PDGF-BB (Thermo) and/or recombinant mouse PDGF-D (R&D Systems) for 16-24 hours at 37 degrees C. Different concentrations of antibodies were pre-incubated with each ligand (10 ng/mL of mouse PDGF-BB and 100 ng/mL of mouse PDGF-D) for 30 minutes at room temperature before treatment to cells. DNA synthesis during the last 3 hours of culture was determined based on 5-bromo-2′-deoxyuridine (BrdU) incorporation using Cell Proliferation ELISA BrdU kit (Roche Applied Science) according to the manufacturer's procedure.

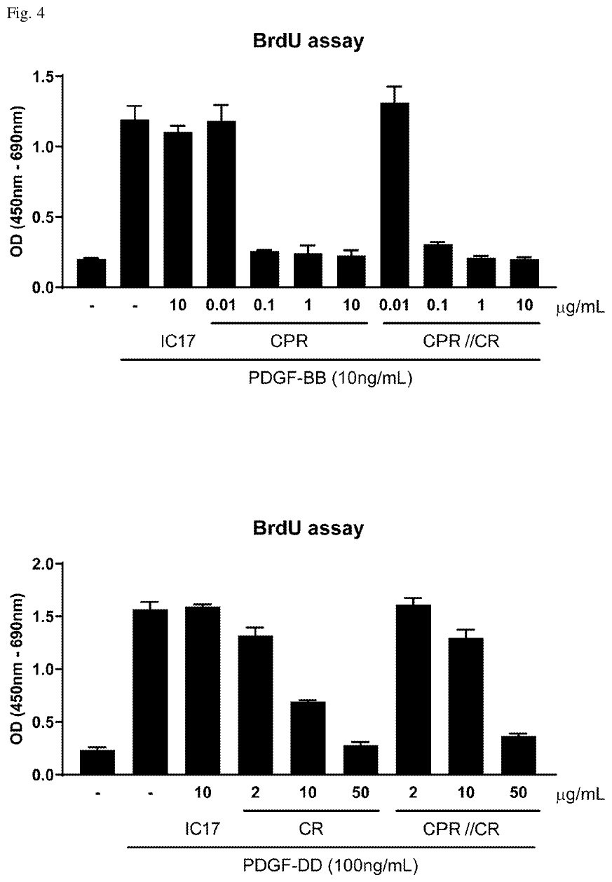

As shown in FIG. 4, CPR (i.e. CPR001) and CPR//CR inhibited PDGF-B-induced cell proliferation, and CR (i.e. CR002) and CPR//CR inhibited PDGF-D-induced cell proliferation of NIH3T3 cells.

### 2.4 NRP1-Fc Inhibited PDGF-D-Induced PDGFR Beta Phosphorylation in Human Fibroblast

Neutralization of recombinant human PDGF-D by NRP1-Fc was tested in human lung fibroblast cell line IMR90 by measuring PDGFR beta phosphorylation.

IMR90 cells were cultured and seeded onto 12-well plates and serum-starved under 0.1% FBS condition overnight. Cells were treated with NRP1-Fc or antibodies and recombinant human PDGF-D (R&D Systems) for 5 minutes at 37 degrees C. 10 g/mL of NRP1-Fc (Sino Biological) or antibodies were pre-incubated with 10 ng/mL of PDGF-D for 30 minutes at room temperature before treatment to cells. Phosphorylation of the PDGFR beta was measured using PathScan (registered trademark) Phospho-PDGF Receptor beta (Tyr751) ELISA kit (Cell Signaling Technology) according to the manufacturer's procedure.

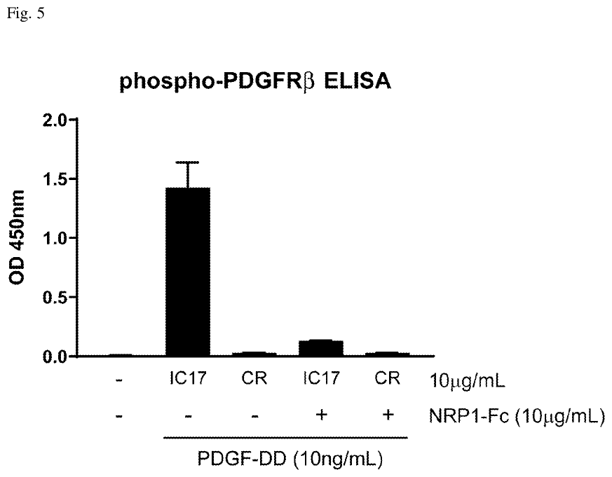

As shown in FIG. 5, NRP1-Fc inhibited PDGF-D-induced PDGFR beta phosphorylation in IMR90 cells. IC17 was used as a negative control and CR (i.e. CR002) was used as positive control for the assay. Results demonstrate that an inhibitor such as a decoy NRP1 molecule (NRP1-Fc) or an antibody that blocks/inhibits PDGF-D binding to NRP1, could inhibit PDGF-D induced PDGFR-beta signalling.

### 2.5 Combination Treatment of NRP1-Fc and Anti-PDGF-D Antibody Inhibited PDGF-D-Induced Proliferation of Human Fibroblast

Neutralization of recombinant human PDGF-D by NRP1-Fc and anti-PDGF-D antibody (CR002) was tested in human lung fibroblast cell line IMR90 by BrdU incorporation.

IMR90 cells were cultured and seeded onto 96-well plates and serum-starved under 0.1% FBS condition overnight. Cells were treated with NRP1-Fc (Sino Biological) and/or antibodies and recombinant human PDGF-D (R&D Systems) for 16-24 hours at 37 degrees C. 10 micro-g/mL of NRP1-Fc and/or antibodies were pre-incubated with 10 ng/mL of PDGF-D for 30 minutes at room temperature before treatment to cells. DNA synthesis during the last 3 hours of culture was determined based on 5-bromo-2′-deoxyuridine (BrdU) incorporation using Cell Proliferation ELISA BrdU kit (Roche Applied Science) according to the manufacturer's procedure.

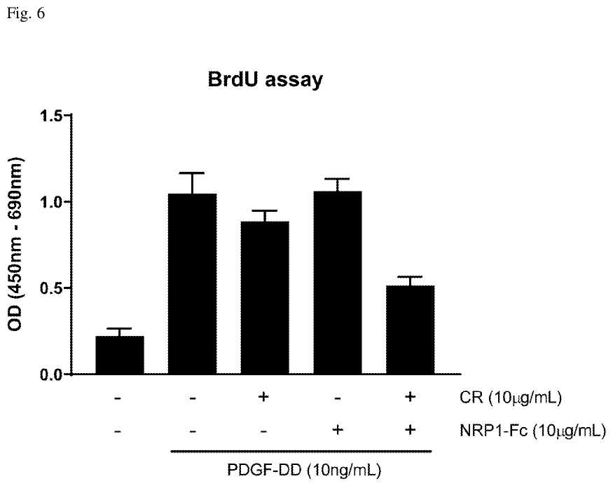

As shown in FIG. 6, combination treatment of NRP1-Fc and CR (i.e. CR002) showed synergistic inhibition of cell proliferation in IMR90. Results suggest that an antigen-binding molecule (e.g. antibody) that specifically binds to PDGF-D and blocks its interaction with Neuropilin 1 (NRP1) and also blocks/inhibits PDGF-D binding to PDGFR, could be developed to effectively inhibit the NRP1-PDGF-D induced signalling. Such antigen-binding molecule is expected to show enhanced inhibition of PDGF-D mediated signalling compared to antigen-binding molecule (e.g. antibody) that specifically binds to PDGF-D but does not block its interaction with NRP1.

Methods of obtaining an antibody that specifically binds to PDGF-D and blocks/inhibits its interaction with NRP1 and also blocks/inhibits PDGF-D binding to PDGFR include known antigen immunization followed by evaluation and screening for inhibition of NRP1-PDGF-D interaction using well-known method such as ELISA, Octet, Biacore and/or ECL and so on.

### Example 3

### Assessment of Anti-PDGF Antibodies in In Vivo Models

### 3.1 Anti-PDGF Antibodies Prevented Kidney Fibrosis in UUO Mouse Model

The in vivo efficacy of monoclonal antibodies CPR001 and CR002 was evaluated in UUO (unilateral ureteral obstruction) mouse model which develops progressive renal fibrosis.

Specific pathogen-free C57BL/6J male mice of 5 weeks of age were purchased from CLEA Japan Inc. (Shiga, Japan) and were acclimated for 2 weeks before the start of treatments. Animals were maintained at 20-26 degrees C. with a 12:12 h light/dark cycle and fed with a commercial standard diet (#CE-2; CLEA Japan Inc., Shizuoka, Japan) and tap water ad libitum.

UUO surgery was operated under isoflurane anesthetized condition. The left side of the abdomen was shaved, and a vertical incision was made through the skin. A second incision was made through the peritoneum and that skin was also retracted to reveal the kidney. Using forceps, the kidney was brought to the surface and the left ureter was tied with surgical silk twice, below the kidney. The ligated kidney was placed gently back into its correct anatomical position then peritoneum and skin were sutured. Analgesic agent was given to reduce animal affliction. In sham operated group, peritoneum and skin were only incised and sutured.

All monoclonal antibodies were administered by intravenous injection once, before the surgical operation. For sham operated group, anti-KLH antibody IC17 was administered. Antibodies were administered at 50 mg/kg. Anti-PDGF-B antibody CPR001, anti-PDGF-D antibody CR002 (as described in WO2007059234), and their combination were administered. Anti-KLH antibody IC17 was used as negative control in this study (50 mg/kg). The combinational treatment group was treated with CPR001 (50 mg/kg) and CR002 (50 mg/kg). The animals were weighed and then killed by exsanguination under isoflurane anaesthesia on day 7. Blood samples were collected from the heart cavities or the postcaval vein and maintained at −80 degrees C. until assayed. The kidney was quickly removed. Part of the kidney tissue was snap-frozen in liquid nitrogen or on dry ice for molecular analyses.

Total RNA was extracted from kidney tissues using RNeasy Mini Kit (Qiagen). Mouse mitochondrial ribosomal protein L19 (MRPL19) was used as the endogenous reference for each sample. Relative mRNA expression values were calculated using double delta Ct analysis.

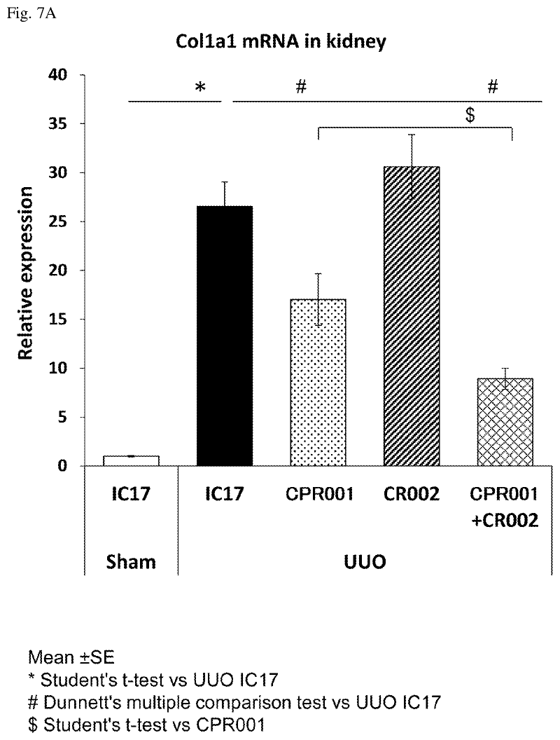

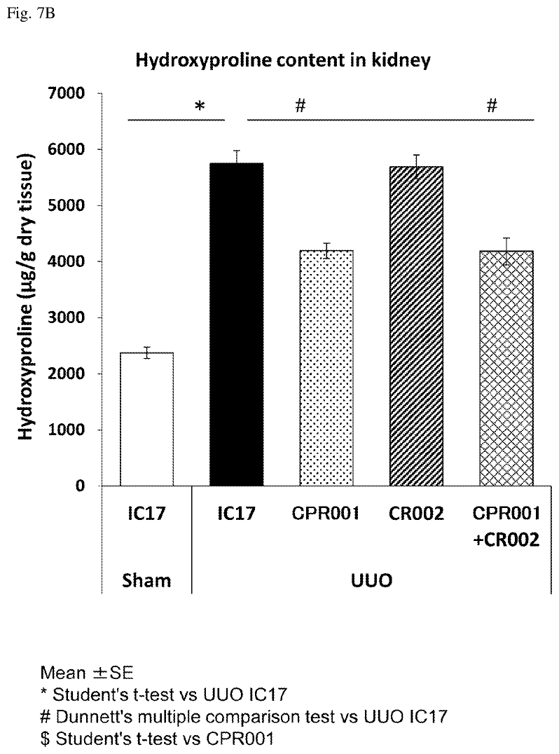

As shown in FIG. 7A, the antibodies' inhibitory activity against kidney fibrosis were evaluated by measuring collagen type 1 alpha 1 mRNA levels in kidney. UUO mice showed significant increase in collagen mRNA level, and CPR001 solo treatment resulted in reduction in collagen mRNA level. Combinational treatment group showed significantly greater reduction compared to CPR001 solo treatment.

The amount of hydroxyproline, which is one of the amino acids included in collagen, in kidney was measured to evaluate the extracellularmatrix deposition to the tissue. Wet kidney tissues were dried up at 110 degrees C. for 3 hours and weighed. Then, 6N HCl (100 micro-L/1 mg dry tissue) was added to the dried tissue and these were boiled overnight. Samples were cleaned up by filtration and 10 micro-L of each sample was plated onto a 96-well plate. The plate with samples was dried out at room temperature overnight and hydroxyproline was measured using hydroxyproline assay kit (BioVision).

As shown in FIG. 7B, significant increase in hydroxyproline content was observed in disease-induced kidney. CPR001 solo treatment and combinational treatment with CR002 inhibited kidney fibrosis. Data is presented as mean+/−standard error of the mean (SEM). Statistical analysis was performed using Student's t-test or Dunnett's multiple comparison test. When P values were <0.05, differences were considered significant.

### 3.2 Anti-PDGF Antibodies Ameliorated Kidney Function and Prevented Kidney Fibrosis in Alport Mouse Model

The in vivo efficacy of monoclonal antibodies CPR001 and CR002 was evaluated in mice deficient for the Col4a3 gene, the so-called Alport mouse model which develops progressive renal glomerular and interstitial fibrosis.

Specific pathogen-free Col4a3 knockout male mice and C57BL/6J male mice of 7 weeks of age were purchased from CLEA Japan Inc. (Shizuoka, Japan) and were acclimated for 3 weeks before the start of treatments. Animals were maintained at 20-26 degrees C. with a 12:12 h light/dark cycle and fed with a commercial standard diet (#CE-2; CLEA Japan Inc., Shizuoka, Japan) and tap water ad libitum.

Blood samples were collected from the jugular vein or the postcaval vein every 2 weeks during the study period. Plasma samples were prepared and maintained at −80 degrees C. until assayed. Twenty-hour urine was collected every 2 weeks during the study period. Plasma and urine biochemistry including creatinine and cystatin C was measured by autoanalyzer TBA-120FR (CANON MEDICAL SYSTEMS, Tochigi, Japan). Alport mice were allocated to diseased control group, CPR001 group, CR002 group, and combinational treatment group on the basis of 12-week age biochemistry (urine albumin/creatinine ratio, plasma creatinine, plasma urea nitrogen) and body weight.

All monoclonal antibodies were administered subcutaneously twice per week from 14 weeks to 22 weeks of age. To the wild type (C57BL/6J) and diseased control groups, anti-KLH antibody IC17 was administered. Antibodies were administered at 50 mg/kg. Anti-PDGF-B antibody CPR001, anti-PDGF-D antibody CR002 (as described in WO2007059234), and their combination were administered. Anti-KLH antibody IC17 was used as negative control in this study. The combination group was treated with CPR001 (50 mg/kg) and CR002 (50 mg/kg). The animals were killed by exsanguination under isoflurane anaesthesia at 22-week age. The kidney was quickly removed. Part of the kidney tissue was snap-frozen in liquid nitrogen or on dry ice for molecular analyses.

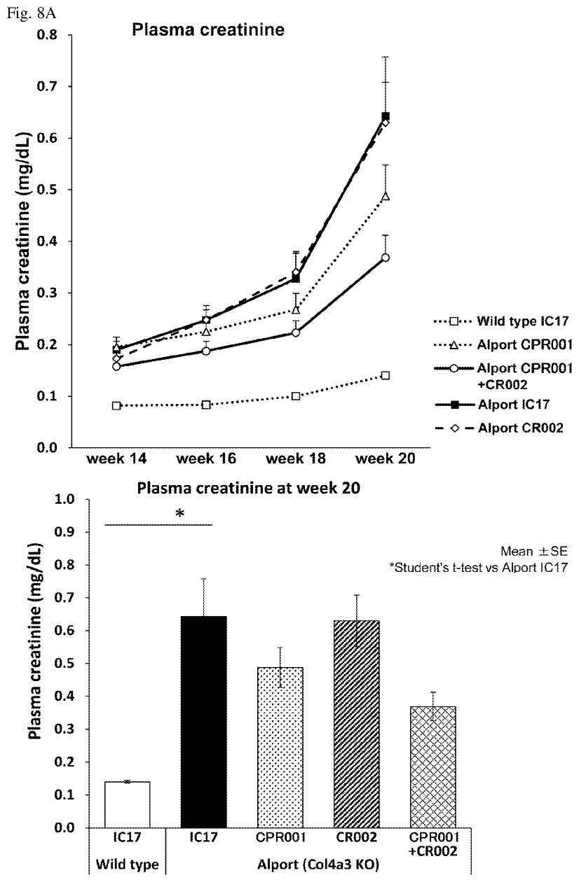

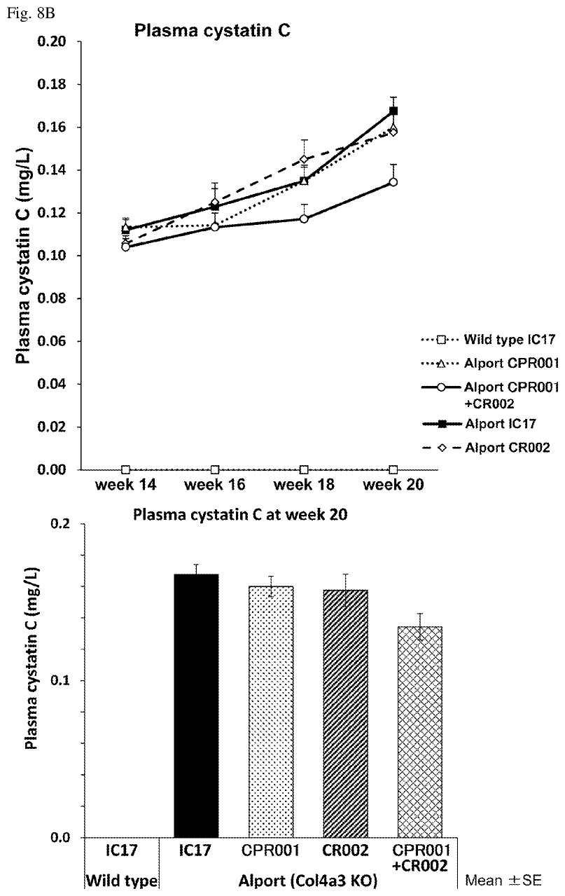

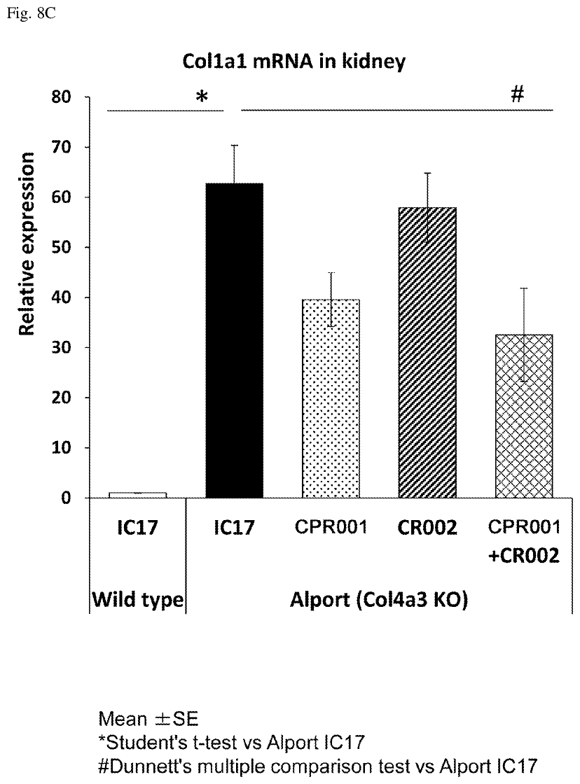

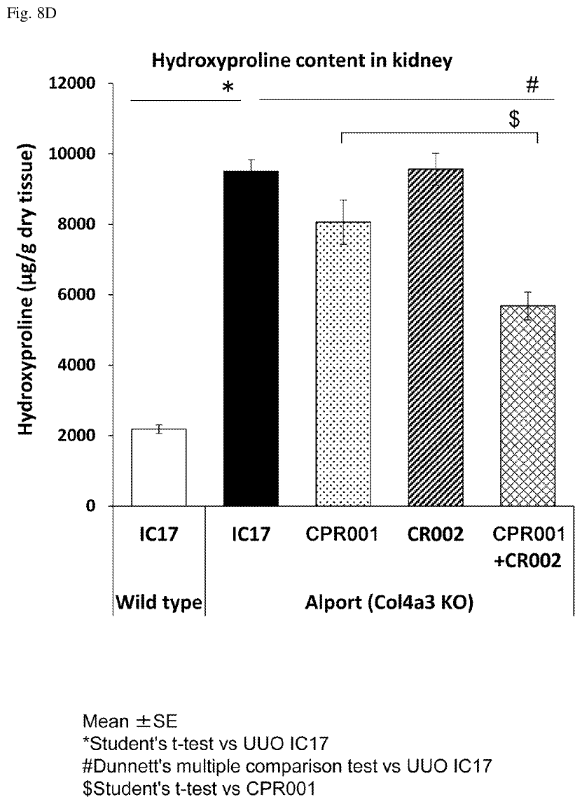

As shown in FIG. 8A, Alport mice showed significant increase in plasma creatinine concentration. CPR001-treated group and combinational treatment group showed reduction in plasma creatinine concentration.

As shown in FIG. 8B, Alport mice showed significant increase in plasma cystatin C concentration. Combinational treatment group showed reduction in plasma cystatin C concentration.

Total RNA was extracted from kidney tissues using RNeasy Mini Kit (Qiagen). Mouse mitochondrial ribosomal protein L19 (MRPL19) was used as the endogenous reference for each sample. Relative mRNA expression values were calculated using double delta Ct analysis.

As shown in FIG. 8C, the inhibitory activity of the antibodies against kidney fibrosis was evaluated based on collagen type 1 alpha 1 mRNA levels in kidney. Alport mice showed significant increase in collagen mRNA level and CPR001-treated group and combinational treatment group showed reduction.

The amount of hydroxyproline, which is one of the amino acids included in collagen, in kidney was measured to evaluate the extracellularmatrix deposition to the tissue. Wet kidney tissues were dried up at 110 degrees C. for 3 hours and weighed. Then, 6N HCl (100 micro-L/1 mg dry tissue) was added to the dried tissue and these were boiled overnight. Samples were cleaned up by filtration and 10 micro-L of each sample was plated onto a 96-well plate. The plate with samples was dried out at room temperature overnight and hydroxyproline was measured using hydroxyproline assay kit (BioVision).

As shown in FIG. 8D, significant increase in hydroxyproline content was observed in disease-induced kidney. In CPR001-treated group and combinational treatment group, kidney fibrosis was inhibited. Combinational treatment group showed significant reduction in hydroxyproline content compared to CPR001 solo treatment. Data is presented as mean+/−standard error of the mean (SEM). Statistical analysis was performed using Student's t-test or Dunnett's multiple comparison test. When P values were <0.05, differences were considered significant.

### 3.3 Anti-PDGF Antibodies Prevented Liver Fibrosis in CDAHFD Model

The in vivo efficacy of monoclonal antibodies CPR001 and CR002 were evaluated in CDAHFD (choline-deficient, L-amino acid-defined, high-fat diet) induced mouse NASH/liver fibrosis model.

All experimental animal care and handling were performed in accordance with the recommendations in the Guidelines for the Care and Use of Laboratory Animals at Chugai Pharmabody Research Pte. Ltd.

Specific pathogen-free C57BL/6NTac male mice of 6 weeks of age were purchased from Invivos Pte Ltd (Singapore) and were acclimated for 1 week before the start of treatments. Animals were maintained at 20-26 degrees C. with a 12:12 h light/dark cycle and fed with a commercial standard diet (5P75; PMI Nutrition INT'L (LabDiet), Missouri, United States) and tap water ad libitum.

The test diet, a choline-deficient, L-amino acid-defined, high-fat diet (CDAHFD; #A06071302), was purchased from Research Diets (New Brunswick, N.J., USA). During the study, 4 groups were fed with CDAHFD (n=8), and 1 group was fed with 5P75 as a normal control group.

All monoclonal antibodies were administered by intravenous injection once per week from 1 week to 3 weeks after disease induction. Antibodies were administered at 50 mg/kg. Anti-PDGF-B antibody CPR001, anti-PDGF-D antibody CR002 (as described in WO2007059234), and their combination were administered. Anti-KLH antibody IC17 was used as negative control in this study. The combinational treatment group was treated with CPR001 (50 mg/kg) and CR002 (50 mg/kg). The animals were weighed and then killed by exsanguination under isoflurane anaesthesia on Day 21. Blood samples were collected from the heart cavity or the postcaval vein and maintained at −80 degrees C. until assayed. The liver was quickly removed and weighed. Part of the liver tissue was snap-frozen in liquid nitrogen or on dry ice for molecular analyses.

Total RNA was extracted from liver tissues using RNeasy Mini Kit (Qiagen), and cDNA was synthesized using a TaqMan (registered trademark) Gene Expression Cells-to-CT Kit (Life Technologies). Gene expression was measured using the QuantStudio™ 12 K Flex Real-Time PCR System (ThermoFisher). Primers and Taq-Man probes for genes were purchased from Applied Biosystems. Mouse glyceraldehyde 3-phosphate dehydrogenase (GAPDH) was used as the endogenous reference for each sample. Relative mRNA expression values were calculated using double delta Ct analysis.

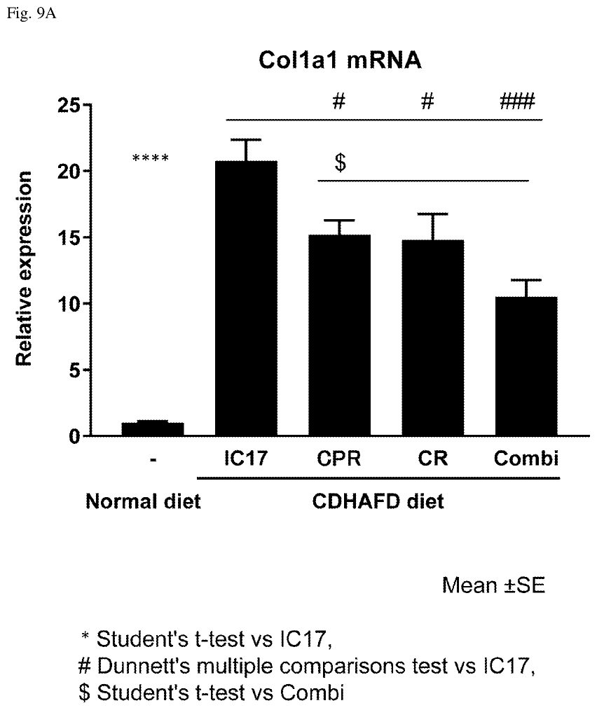

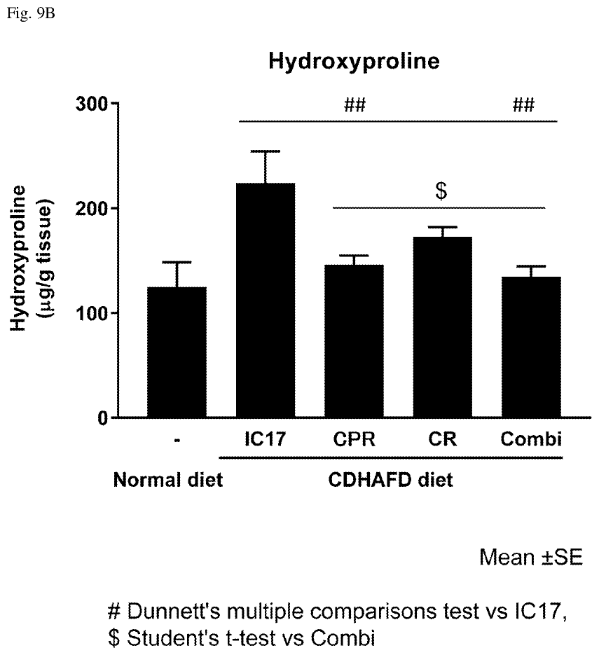

As shown in FIG. 9A, the antibodies' inhibitory activity against liver fibrosis was evaluated based on the amount of collagen type 1 alpha 1 mRNA in liver. CDAHFD mice showed significant increase in collagen mRNA level and all three treatment groups showed the reduction. Combinational treatment group showed significant reduction compared to CPR001 solo treatment.

The amount of hydroxyproline, which is one of the amino acids included in collagen, in liver was measured to evaluate the extracellularmatrix deposition to the tissue. Wet liver tissues were homogenized in distilled H₂O (50 micro-L/10 mg wet tissue). Then, equal amount of 12N HCl was added to the homogenized tissues and these were boiled at 110 degrees C. overnight. Samples were cleaned up by filtration and 10 micro-L of each sample was plated onto a 96-well plate. The plate with samples was dried out at room temperature and hydroxyproline was measured using hydroxyproline assay kit (BioVision).

As shown in FIG. 9B, increased hydroxyproline content was observed in disease-induced liver and all three treatment groups showed the reduction. Combinational treatment group (CPR001 and CR002) showed significant reduction compared to CR002 solo treatment. Data is presented as mean+/−standard error of the mean (SEM). Statistical analysis was performed using Student's t-test or Dunnett's multiple comparison test. When P values were <0.05, differences were considered significant.

### Example 4

### Assessment of Anti-PDGF-B/D Bi-Specific Antibody in In Vivo Models 4.1 CPR//CR Bi-Specific Antibody Prevented Kidney Fibrosis in UUO Mouse Model

The in vivo efficacy of monoclonal antibody CPR//CR was evaluated in

Unilateral Ureteral Obstruction (UUO) mouse model which develops progressive renal fibrosis.

Specific pathogen-free C57BL/6J male mice of 7 weeks of age were purchased from CLEA Japan Inc. (Shiga, Japan) and were acclimated for 1 week before the start of treatments. Animals were maintained at 20 to 26 degrees C. with a 12:12 h light/dark cycle and fed with a commercial standard diet (#CE-2; CLEA Japan Inc., Shizuoka, Japan) and tap water ad libitum.

UUO surgery was operated under isoflurane anesthetized condition. The left side of the abdomen was shaved, and a vertical incision was made through the skin. A second incision was made through the peritoneum and that skin was also retracted to reveal the kidney. Using forceps, the kidney was brought to the surface and the left ureter was tied with surgical silk twice, below the kidney. The ligated kidney was placed gently back into its correct anatomical position then peritoneum and skin were sutured. Analgesic agent was given to reduce animal affliction. In sham operated group, peritoneum and skin were only incised and sutured.

All monoclonal antibody was administered by intravenous injection once before the surgical operation. Sham operated group was administered anti-KLH antibody IC17. CPR//CR bi-specific antibody against PDGF-B and PDGF-D was administered. Anti-KLH antibody IC17 was used as negative control in this study. Antibodies were administered at 50 mg/kg. The animals were weighed and then killed by exsanguination under isoflurane anaesthesia on day 7. Blood samples were collected from the heart cavities or the postcaval vein and maintained at −80 degrees C. until assayed. The kidney was quickly removed. Part of the kidney tissue was snap-frozen in liquid nitrogen or on dry ice for molecular analyses.

Total RNA was extracted from kidney tissues using RNeasy Mini Kit (Qiagen). Mouse mitochondrial ribosomal protein L19 (MRPL19) was used as the endogenous reference for each sample. Relative mRNA expression values were calculated using double delta Ct analysis.

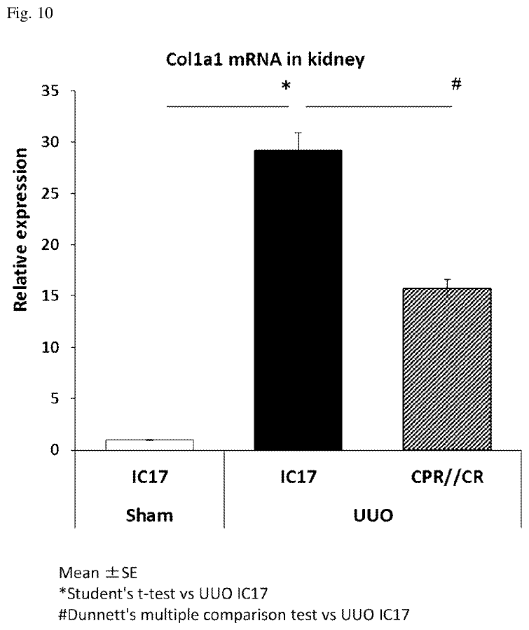

As shown in FIG. 10, the antibody inhibitory activity against kidney fibrosis was evaluated by collagen type 1 alpha 1 mRNA in kidney. UUO mice showed significant increase in collagen mRNA level. CPR//CR group showed significant reduction compared to the diseased control group (UUO IC17).

The amount of hydroxyproline, which is one of the amino acids included in collagen, in kidney was measured to evaluate the extracellularmatrix deposition to the tissue. Wet kidney tissues were dried up at 110 degrees C. for 3 hours and weighed. Then, 6N HCl (100 micro-L/1 mg dry tissue) was added to the dried tissue and these were boiled overnight. Samples were cleaned up by filteration and 10 micro-L of each sample was plated onto a 96-well plate. The plate with samples was dried out at room temperature overnight and hydroxyproline was measured using hydroxyproline assay kit (BioVision).

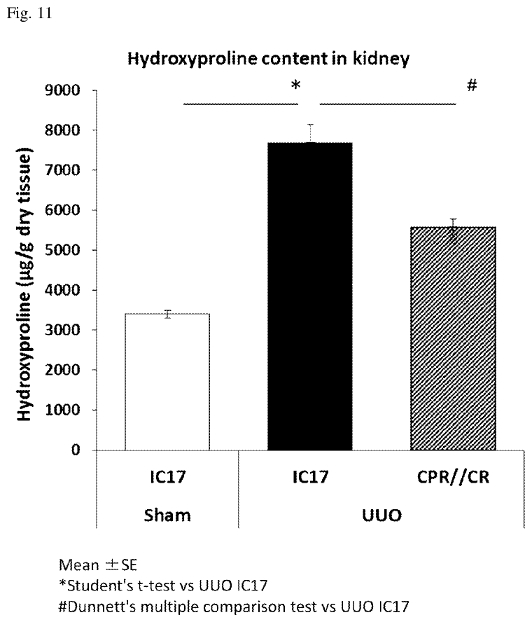

A shown in FIG. 11, significant increase in hydroxyproline content was observed in disease induced kidney. CPR//CR treatment inhibited kidney fibrosis.

Data are presented as mean+/−standard error of the mean (SEM). Statistical analysis was performed using Student's t-test or Dunnett's multiple comparison test. When P values were <0.05, differences were considered significant.

### 4.2 CPR//CR Bi-Specific Antibody Ameliorated Kidney Function and Prevented Kidney Fibrosis in Alport Mouse Model

The in vivo efficacy of monoclonal antibody CPR//CR was evaluated in mice deficient for the Col4a3 gene, the so-called Alport mouse model which develops progressive renal glomerular and interstitial fibrosis.

Specific pathogen-free Col4a3 knockout male mice and C57BL/6J male mice of 7 weeks of age were supplied from CLEA Japan Inc. (Shizuoka, Japan) and were acclimated for 3 weeks before the start of treatments. Animals were maintained at 20 to 26 degrees C. with a 12:12 h light/dark cycle and fed with a commercial standard diet (#CE-2; CLEA Japan Inc., Shizuoka, Japan) and tap water ad libitum.

Blood samples were collected from the jugular vein or the postcaval vein every 2 weeks during the study period. Plasma samples were prepared and maintained at −80 degrees C. until assayed. Twenty-hour urine was collected every 2 weeks during the study period. Plasma and urine biochemistry including creatinine and cystatin C was measured by autoanalyzer TBA-120FR (CANON MEDICAL SYSTEMS, Tochigi, Japan). Alport mice were allocated to diseased control group, CPR001 group, CR002 group, and combinational treatment group on the basis of 12-week age biochemistry (urine albumin/creatinine ratio, plasma creatinine, plasma urea nitrogen) and body weight.

All monoclonal antibodies were administered subcutaneously twice per week from 14 weeks to 20 weeks of age. To the wild type (C57BL/6N) and diseased control groups, anti-KLH antibody IC17 was administered. Antibodies, including bi-specific antibody CPR//CR against PDGF-B and PDGF-D, were administered at 50 mg/kg. Anti-KLH antibody IC17 was used as negative control in this study. The animals were killed by exsanguination under isoflurane anaesthesia at 20-week age. The kidney was quickly removed. Part of the kidney tissue was snap-frozen in liquid nitrogen or on dry ice for molecular analyses.

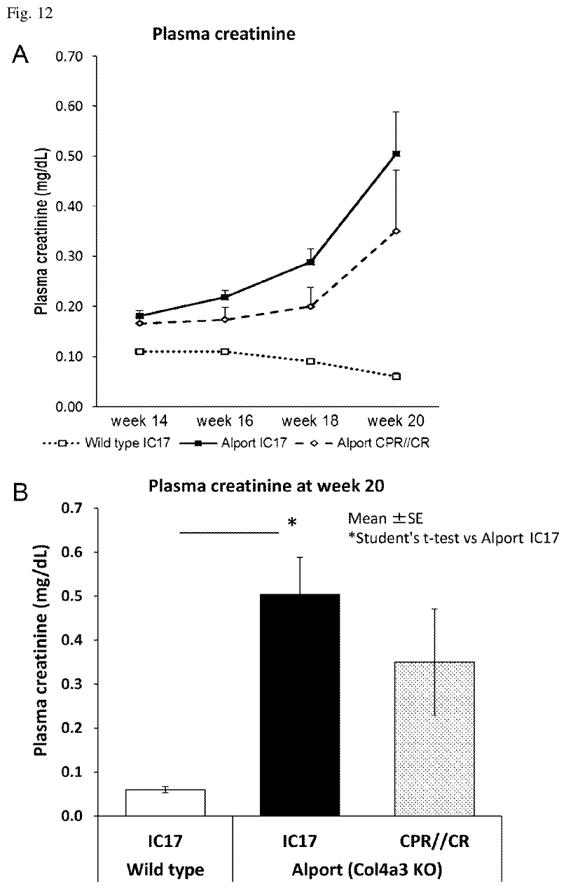

As shown in FIG. 12, Alport mice showed significant increase in plasma creatinine concentration and CPR//CR-treated group showed reduction in plasma creatinine concentration.

Total RNA was extracted from kidney tissues in the same manner as in Example 4.1. Mouse mitochondrial ribosomal protein L19 (MRPL19) was used as the endogenous reference for each sample. Relative mRNA expression values were calculated using double delta Ct analysis.

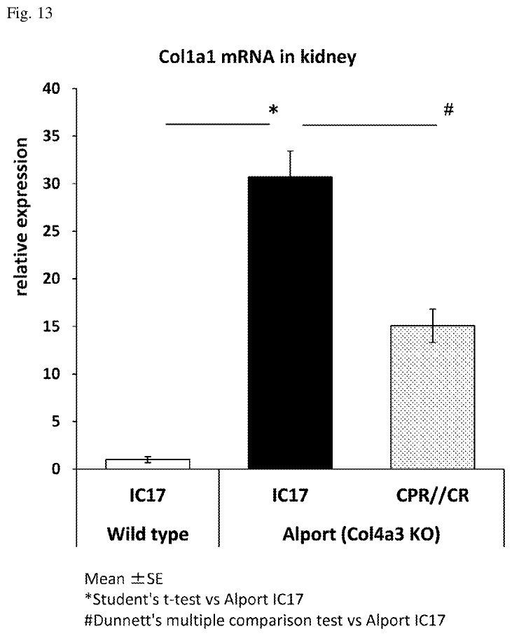

As shown in FIG. 13, the inhibitory activity of the antibodies against kidney fibrosis was evaluated based on collagen type 1 alpha 1 mRNA levels in kidney. Alport mice showed significant increase in collagen mRNA level and CPR//CR showed significant reduction.

The amount of hydroxyproline, which is one of the amino acids included in collagen, in kidney was measured to evaluate the extracellularmatrix deposition to the tissue. Wet kidney tissues were dried up at 110 degrees C. for 3 hours and weighed. Then, 6N HCl (100 micro-L/1 mg dry tissue) was added to the dried tissue and these were boiled overnight. Samples were cleaned up by filteration and 10 micro-L of each sample was plated onto a 96-well plate. The plate with samples was dried out at room temperature overnight and hydroxyproline was measured using hydroxyproline assay kit (BioVision).

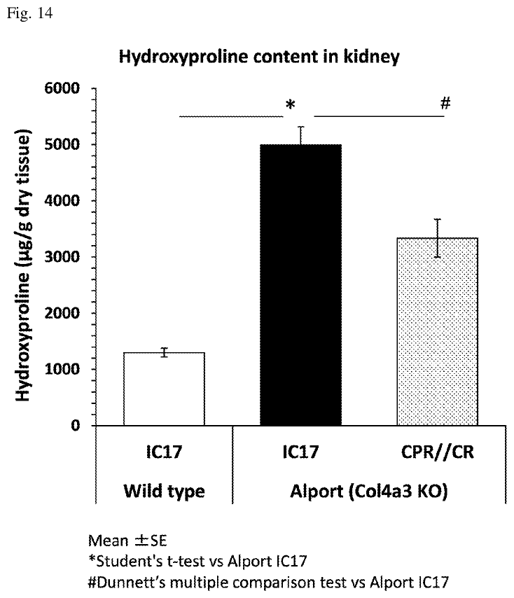

As shown in FIG. 14, significant increase in hydroxyproline content was observed in disease induced kidney and CPR//CR inhibited kidney fibrosis.

Data are presented as mean+/−standard error of the mean (SEM). Statistical analysis was performed using Student's t-test or Dunnett's multiple comparison test. When P values were <0.05, differences were considered significant.

### 4.3 CPR//CR Bi-Specific Antibody Prevented Liver Fibrosis in CDAHFD Model

The in vivo efficacy of monoclonal antibody CPR//CR was evaluated in CDAHFD (choline-deficient, L-amino acid-defined, high-fat diet) induced mouse NASH/liver fibrosis model.

All experimental animal care and handling were performed in accordance with the recommendations in the Guidelines for the Care and Use of Laboratory Animals at Chugai Pharmabody Research Pte. Ltd.

Specific pathogen-free C57BL/6NTac male mice of 6 weeks of age were purchased from Invivos Pte Ltd (Singapore) and were acclimated for 1 weeks before the start of treatments. Animals were maintained at 20 to 26 degrees C. with a 12:12 h light/dark cycle and fed with a commercial standard diet (5P75; PMI Nutrition INT'L (LabDiet), Missouri, United States) and tap water ad libitum.

The test diet, a choline-deficient, L-amino acid-defined, high-fat diet (CDAHFD; #A06071302), was purchased from Research Diets (New Brunswick, N.J., USA). During the study, 4 groups were fed CDAHFD (n=8), and 1 group was fed 5P75 as a normal control group.

All monoclonal antibodies were administered by intravenous injection once per week at day 0 and day 7 during the study. CPR//CR bi-specific antibody against PDGF-B and PDGF-D was administered in different dosage. Anti-KLH antibody IC17 was used as negative control in this study. The animals were weighed and then killed by exsanguination under isoflurane anaesthesia on Day 14. Blood samples were collected from the heart cavity or the postcaval vein and maintained at −80 degrees C. until assayed. The liver was quickly removed and weighed. Part of the liver tissue was snap-frozen in liquid nitrogen or on dry ice for molecular analyses.

Total RNA was extracted from liver tissues using RNeasy Mini Kit (Qiagen), and cDNA was synthesized using a TaqMan (registered trademark) Gene Expression Cells-to-CT Kit (Life Technologies). Gene expression was measured using the QuantStudio™ 12 K Flex Real-Time PCR System (ThermoFisher). Primers and Taq-Man probes for genes were purchased from Applied Biosystems. Mouse glyceraldehyde 3-phosphate dehydrogenase (GAPDH) was used as the endogenous reference for each sample. Relative mRNA expression values were calculated using double delta Ct analysis.

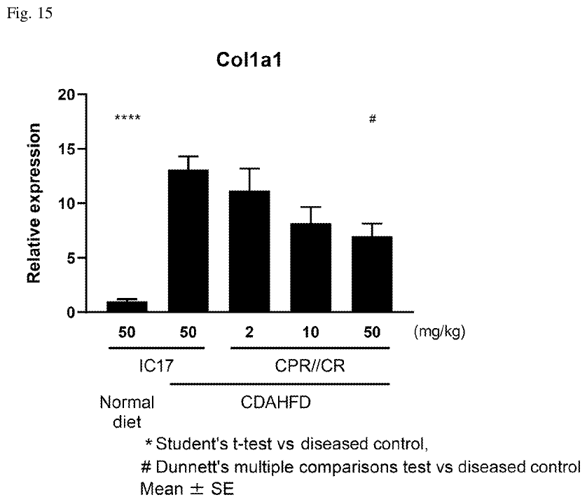

As shown in FIG. 15, the antibodies' inhibitory activity against liver fibrosis was evaluated based on the amount of collagen type 1 alpha 1 mRNA in liver. CDAHFD mice showed significant increase in collagen mRNA level and CPR//CR treatment groups showed the reduction.

Plasma aspartate aminotransferase (AST) and alanine aminotransferase (ALT), which are liver enzymes, were measured to evaluate the liver disease and damage. Plasma AST and ALT were measured according to the kit instruction (BioVision #K752 and #1(753).

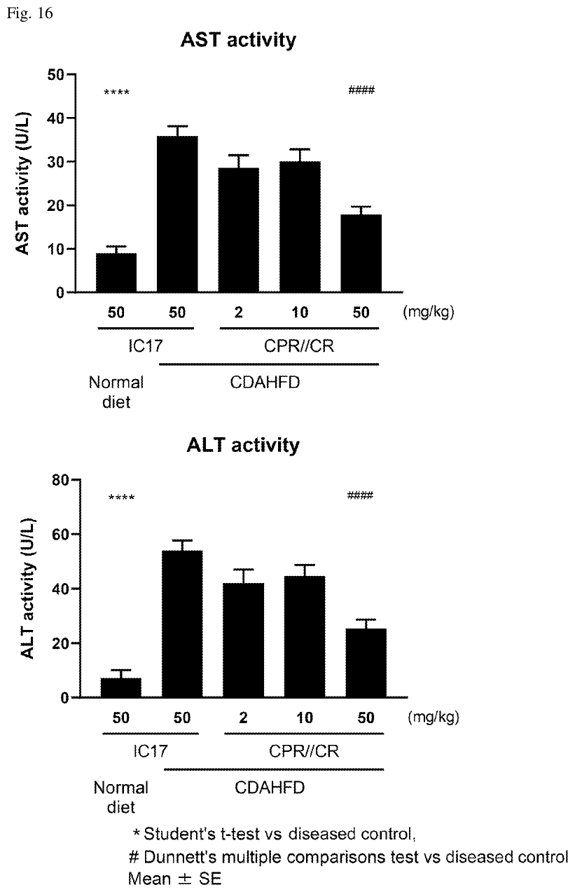

As shown in FIG. 16, increased plasma ALT and AST were observed in disease induced group and CPR//CR treatment groups showed reduction.

Data are presented as mean+/−standard error of the mean (SEM). Statistical analysis was performed using Student's t-test or Dunnett's multiple comparison test. When P values were <0.05, differences were considered significant.

### Reference Example 1

### Binding Activity Assessment of Anti-PDGF Antibodies

### 1.1 Biacore Analysis for Binding Affinity Evaluation of Anti-PDGF-B/D Bispecific Antibody

Binding affinity (KD) of anti-PDGF antibodies (CPR001, CR002, or CPR//CR bi-specific antibody) binding to human PDGF-B or PDGF-D at pH 7.4 was determined at 25 degrees C. using Biacore T200 instrument (GE Healthcare) (Table 3). Anti-human Fc (GE Healthcare) was immobilized onto all flow cells of a CM4 sensor chip using amine coupling kit (GE Healthcare). All antibodies and analytes were prepared in PBS-NET (10 mM phosphate, 287 mM NaCl, 2.7 mM KCl, 3.2 mM EDTA, 0.01% P20, 0.005% NaN₃, pH 7.4). Each antibody was captured onto the sensor surface by anti-human Fc. Antibody capture levels were aimed at 100 resonance unit (RU). Recombinant human PDGF-B or PDGF-D was injected at 1 and 5 nM prepared by five-fold serial dilution, followed by dissociation. Sensor surface was regenerated each cycle with 3M MgCl₂. Binding affinity was determined by processing and fitting the data to 1:1 binding model using Biacore T200 Evaluation software, version 2.0 (GE Healthcare).

TABLE 3
The binding affinity (KD) of anti-PDGF-B or anti-PDGF-D
antibodies binding to human PDGF-B or human PDGF-D.

| Antibody Name | Antigen | KD (M) |
|---|---|---|
| CPR001-GL-F1332m | hPDGF-B | 2.02E−11 |
| CR002-F1332m | hPDGF-B | n.d. |
| CPR001GLVH/xIC17-SG181v14hk | hPDGF-B | 1.65E−11 |
| CPR001GL/xCR002-SG181v14hk | hPDGF-B | 2.11E−11 |
| CPR001-GL-F1332m | hPDGF-D | n.d. |
| CR002-F1332m | hPDGF-D | 4.03E−14* |
| CPR001GLVH/xIC17-SG181v14hk | hPDGF-D | n.d. |
| CPR001GL/xCR002-SG181v14hk | hPDGF-D | 5.34E−13* |

Note:
n.d. KD cannot be determined due to low binding response.
*strong binders, slow off rate <1E−05, KD cannot be uniquely determined.
“E−n” means “−n^{th} power of 10” or “10⁻ⁿ” (for example, 1.0E−6 means 1.0 × 10⁻⁶).

1.2 Biacore Premix Competition Assay for hPDGF-D/hPDGFR Beta/Anti-PDGF-D Antibody

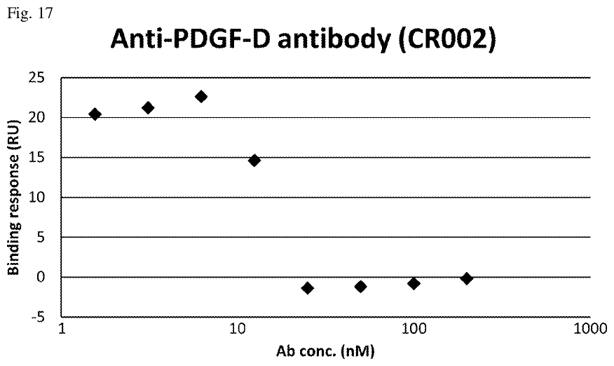

Competition binding of anti-PDGF-D antibody (CR002) and hPDGFR beta against hPDGF-D was evaluated by premix competition assay. Anti-PDGF-D antibody (CR002) with mouse Fc (final concentration of 200 nM to 1.6 nM, two-fold serial dilution) was mixed with human PDGF-D (final concentration: 10 nM) in PBS-NET and incubated for 1 hour at 25 degrees C. to reach equilibrium. Each anti-PDGF-D antibody (CR002) and human PDGF-D dilution mixture was then injected over the surface of a CM4 chip, covered with human PDGFR beta-hlgG1 captured by Anti-human Fc (GE Healthcare). Sensor surface was regenerated each cycle with 3M MgCl₂. Binding response at 95 sec was recorded and graph for binding response vs concentration of anti-PDGF-D antibody (CR002) was plotted. Binding of PDGF-D to PDGFR beta was blocked with increased concentration of anti-PDGF-D antibody (CR002) as shown in FIG. 17.

1.3 Biacore Competition Assay for hPDGF-D/hNRP1-Fc/Anti-PDGF-D Antibody

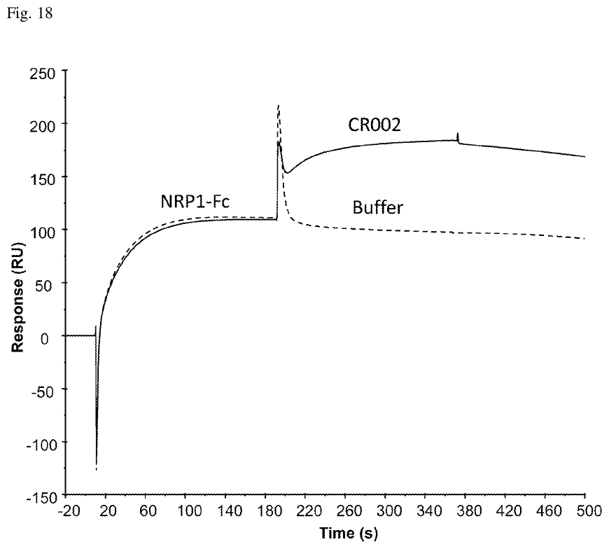

Biacore in-tandem blocking assay was performed to characterize binding epitope of anti-PDGF-D antibody (CR002) and hNRP1-Fc against hPDGF-D. The assay was performed on Biacore T200 instrument (GE Healthcare) at 37 degrees C. in HBS-EP+ buffer 10 mM HEPES pH 7.4, 150 mM NaCl, 3 mM EDTA, 0.005% v/v P20. Human PDGF-D was immobilized onto flow cell 4 of a CM4 sensor chip using amine coupling kit (GE Healthcare). Blank immobilization was performed on flow cell 3 and this was used as reference flow cell. Then 100 nM NRP1-Fc at saturating concentration was injected for 3 mM, and following that 1000 nM anti-PDGF-D antibody (CR002) was injected similarly as competing partner. Sensor surface was regenerated each cycle with 3M MgCl₂. As shown in FIG. 18, binding response for CR002 injection which was greater than that observed for buffer injection indicates that CR002 and hNRP-1 bind to different epitopes on hPDGF-D.

Although the foregoing invention has been described in some detail by way of illustration and example for purposes of clarity of understanding, the descriptions and examples should not be construed as limiting the scope of the present invention. The disclosures of all patent and scientific literature cited above are expressly incorporated herein in their entirety by reference.

### SEQUENCE LISTING

1. A multispecific antigen-binding molecule comprising a first antigen-binding domain that binds to PDGF-B, and a second antigen-binding domain that binds to PDGF-D.

2. The multispecific antigen-binding molecule of claim 1, comprising one or more of the following properties:
i) inhibiting PDGF B and PDGF-D binding to PDGFR alpha and/or PDGFR beta;
ii) inhibiting PDGF-B and PDGF-D mediated phosphorylation of PDGFR alpha and/or PDGFR beta;
iii) inhibiting PDGF-B and PDGF-D induced dimerization of PDGFR alpha and/or PDGFR beta;
iv) inhibiting PDGF-B and PDGF-D induced mitogenesis of cells displaying PDGFR alpha and/or PDGFR beta; and
v) not binding to PDGF-A and/or PDGF-C.

3. The multispecific antigen-binding molecule of claim 1 or 2, wherein the antigen-binding molecule is an antibody, preferably a monoclonal antibody, a chimeric antibody, a humanized antibody, a human antibody, or a fragment thereof.

4. The multispecific antigen-binding molecule of any one of claims 1 to 3, wherein the first antigen-binding domain that binds to PDGF-B is:
(a) an antigen-binding domain that comprises a VH comprising the amino acid sequence of SEQ ID NO: 1; and a VL comprising the amino acid sequence of SEQ ID NO: 2;
(b) an antigen-binding domain that comprises a VH comprising the CDR-H1 amino acid sequence of SEQ ID NO: 5, the CDR-H2 amino acid sequence of SEQ ID NO: 6, and the CDR-H3 amino acid sequence of SEQ ID NO: 7; and a VL comprising the CDR-L1 amino acid sequence of SEQ ID NO: 8, the CDR-L2 amino acid sequence of SEQ ID NO: 9, and the CDR-L3 amino acid sequence of SEQ ID NO: 10;
(c) an antigen-binding domain that binds to the same epitope on PDGF-B with any one of the antigen-binding domains of (a) to (b); or
(d) an antigen-binding domain that competes for binding to PDGF-B with any one of the antigen-binding domains of (a) to (b);
and/or
the second antigen-binding domain that binds to PDGF-D is:
(e) an antigen-binding domain that comprises a VH comprising the amino acid sequence of SEQ ID NO: 3; and a VL comprising the amino acid sequence of SEQ ID NO: 4;
(f) an antigen-binding domain that comprises a VH comprising the CDR-H1 amino acid sequence of SEQ ID NO: 11, the CDR-H2 amino acid sequence of SEQ ID NO: 12, and the CDR-H3 amino acid sequence of SEQ ID NO: 13; and a VL comprising the CDR-L1 amino acid sequence of SEQ ID NO: 14, the CDR-L2 amino acid sequence of SEQ ID NO: 15, and the CDR-L3 amino acid sequence of SEQ ID NO: 16;
(g) an antigen-binding domain that binds to the same epitope on PDGF-D with any one of the antigen-binding domains of (e) to (f); or
(h) an antigen-binding domain that competes for binding to PDGF-D with any one of the antigen-binding domains of (e) to (f).

5. The multispecific antigen-binding molecule of any one of claims 1 to 4, further comprising an antibody Fc region with reduced binding activity towards an Fc gamma receptor.

6. The multispecific antigen-binding molecule of any one of claims 1 to 5, for use in the treatment of a fibrotic disease or fibrosis.

7. A method for preventing, treating, or inhibiting a fibrotic disease or fibrosis comprising: administering to a mammalian subject suffering from the fibrotic disease or fibrosis the multispecific antigen-binding molecule of any one of claims 1 to 5.

8. The multispecific antigen-binding molecule for use or the method according to claim 6 or 7, wherein the fibrotic disease or fibrosis is characterized by upregulated PDGF signaling activation.

9. The multispecific antigen-binding molecule for use or the method according to any one of claims 6-8, wherein the fibrotic disease or fibrosis is myocardial fibrosis, pulmonary fibrosis, liver fibrosis, renal fibrosis, skin fibrosis, ocular fibrosis and myelofibrosis, nephritis, progressive renal diseases, IgA nephropathy, mesangial proliferative nephritis, mesangial proliferative glomerulonephritis, mesangiocapillary glomerulonephritis, systemic lupus erythematosus, glomerular nephritis, renal interstitial fibrosis, renal failure, diabetic nephropathy, polycystic kidney disease, alport syndrome, focal segmental glomerular sclerosis, or membranous nephropathy.

10. The multispecific antigen-binding molecule for use or the method according to any one of claims 6-8, wherein the fibrotic disease or fibrosis is kidney fibrosis, preferably characterized by having interstitial fibrosis or glomerulosclerosis.

11. An isolated polynucleotide comprising a nucleotide sequence that encodes the multispecific antigen-binding molecule of any one of claims 1 to 5.

12. An expression vector comprising the polynucleotide according to claim 11.

13. A host cell transformed or transfected with the polynucleotide according to claim 11 or the expression vector according to claim 12.

14. A method of producing an antigen-binding molecule comprising:
(a) identifying one or more antigen-binding domain that binds to PDGF-B;
(b) identifying one or more antigen-binding domain that binds to PDGF-D; and
(c) preparing an antigen-binding molecule comprising the antigen-binding domain identified in (a) and (b).


SEQUENCE LISTING (40 sequences)

SEQ ID NO: 1 - protein, 120 aa, Artificial Sequence
  An artificially synthesized sequence
EVQLLESGGGLVQPGGSLRLSCAASGFTFSSYAMSWVRQAPGKGLEWVSYISDDGSLKYYADSVKGRFTISRDNSKNTLYLQMNSLRAEDTAVYYCAKHPYWYGGQLDLWGQGTLVTVSS

SEQ ID NO: 2 - protein, 106 aa, Artificial Sequence
  An artificially synthesized sequence
SYELTQPPSVSVSPGQTASITCSGDSLGSYFVHWYQQKPGQSPVLVIYDDSNRPSGIPERFSGSNSGNTATLTISGTQAMDEADYYCSAFTHNSDVFGGGTKLTVL

SEQ ID NO: 3 - protein, 125 aa, Artificial Sequence
  An artificially synthesized sequence
QVQLVQSGAEVKKPGASVKVSCKASGYTFTSYDINWVRQATGQGLEWMGWINPNSGNTDYAQKFQGRVTMTRDTSISTAYMELSSLRSEDTAIYYCVRGFGYSYNYDYYYGMDVWGQGTTVTVSS

SEQ ID NO: 4 - protein, 108 aa, Artificial Sequence
  An artificially synthesized sequence
EIVLTQSPGTLSLSPGERATLSCRASQSVSSSYLAWYQQKPGQAPRLLIYATSSRATGIPDRFSGSGSGTDFTLTISRLEPEDFAVYYCQQYGSSPCSFGQGTKLEIK

SEQ ID NO: 5 - protein, 5 aa, Artificial Sequence
  An artificially synthesized sequence
SYAMS

SEQ ID NO: 6 - protein, 17 aa, Artificial Sequence
  An artificially synthesized sequence
YISDDGSLKYYADSVKG

SEQ ID NO: 7 - protein, 11 aa, Artificial Sequence
  An artificially synthesized sequence
HPYWYGGQLDL

SEQ ID NO: 8 - protein, 11 aa, Artificial Sequence
  An artificially synthesized sequence
SGDSLGSYFVH

SEQ ID NO: 9 - protein, 7 aa, Artificial Sequence
  An artificially synthesized sequence
DDSNRPS

SEQ ID NO: 10 - protein, 9 aa, Artificial Sequence
  An artificially synthesized sequence
SAFTHNSDV

SEQ ID NO: 11 - protein, 5 aa, Artificial Sequence
  An artificially synthesized sequence
SYDIN

SEQ ID NO: 12 - protein, 17 aa, Artificial Sequence
  An artificially synthesized sequence
WINPNSGNTDYAQKFQG

SEQ ID NO: 13 - protein, 16 aa, Artificial Sequence
  An artificially synthesized sequence
GFGYSYNYDYYYGMDV

SEQ ID NO: 14 - protein, 12 aa, Artificial Sequence
  An artificially synthesized sequence
RASQSVSSSYLA

SEQ ID NO: 15 - protein, 7 aa, Artificial Sequence
  An artificially synthesized sequence
ATSSRAT

SEQ ID NO: 16 - protein, 9 aa, Artificial Sequence
  An artificially synthesized sequence
QQYGSSPCS

SEQ ID NO: 17 - protein, 240 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 18 - protein, 206 aa, Homo sapiens
IFEAVGAQGTVVLDEESLHVDDLDLFDGDSGLRFAEDLEVSELKVGADLEREGDLGEVPPHAVHESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNW

SEQ ID NO: 19 - protein, 330 aa, Homo sapiens [sequence omitted]

SEQ ID NO: 20 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 22 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 24 - protein, 316 aa, Homo sapiens
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 25 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 26 - protein, 254 aa, Homo sapiens [sequence omitted]

SEQ ID NO: 27 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 28 - DNA, 1125 bp, Homo sapiens
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtag

SEQ ID NO: 29 - DNA, 951 bp, Homo sapiens
atgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatgggggtcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 30 - DNA, 876 bp, Homo sapiens
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaatcccactaatcctgatgaggctgacaaagttggggctgagaacacaatcacctattcacttctcatgcacccggatgctctggaagagcctgatgaccagaaccgtatttag

SEQ ID NO: 31 - DNA, 765 bp, Homo sapiens
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttgtcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 32 - DNA, 702 bp, Homo sapiens
atgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagagcctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttga

SEQ ID NO: 33 - protein, 4 aa, Artificial Sequence
  An artificially synthesized sequence
GGGS

SEQ ID NO: 34 - protein, 4 aa, Artificial Sequence
  An artificially synthesized sequence
SGGG

SEQ ID NO: 35 - protein, 5 aa, Artificial Sequence
  An artificially synthesized sequence
GGGGS

SEQ ID NO: 36 - protein, 5 aa, Artificial Sequence
  An artificially synthesized sequence
SGGGG

SEQ ID NO: 37 - protein, 6 aa, Artificial Sequence
  An artificially synthesized sequence
GGGGGS

SEQ ID NO: 38 - protein, 6 aa, Artificial Sequence
  An artificially synthesized sequence
SGGGGG

SEQ ID NO: 39 - protein, 7 aa, Artificial Sequence
  An artificially synthesized sequence
GGGGGGS

SEQ ID NO: 40 - protein, 7 aa, Artificial Sequence
  An artificially synthesized sequence
SGGGGGG